SEQUENCE LISTING (64 sequences)

SEQ ID NO: 1 - protein, 217 aa, Artificial Sequence
  Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic Polypeptide
SKYGPPCPSCP

SEQ ID NO: 5 - protein, 11 aa, Artificial Sequence
  Synthetic Polypeptide
SKYGPPCPPCP

SEQ ID NO: 6 - protein, 6 aa, Artificial Sequence
  Synthetic Polypeptide
SKYGPP

SEQ ID NO: 7 - protein, 6 aa, Artificial Sequence
  Synthetic Polypeptide
IEGRMD

SEQ ID NO: 8 - protein, 9 aa, Artificial Sequence
  Synthetic Polypeptide
GGGVPRDCG

SEQ ID NO: 9 - protein, 15 aa, Artificial Sequence
  Synthetic Polypeptide
IEGRMDGGGGAGGGG

SEQ ID NO: 10 - protein, 8 aa, Artificial Sequence
  Synthetic Polypeptide
GGGSGGGS

SEQ ID NO: 11 - protein, 12 aa, Artificial Sequence
  Synthetic Polypeptide
GGGSGGGGSGGG

SEQ ID NO: 12 - protein, 14 aa, Artificial Sequence
  Synthetic Polypeptide
EGKSSGSGSESKST

SEQ ID NO: 13 - protein, 4 aa, Artificial Sequence
  Synthetic Polypeptide
GGSG

SEQ ID NO: 14 - protein, 12 aa, Artificial Sequence
  Synthetic Polypeptide
GGSGGGSGGGSG

SEQ ID NO: 15 - protein, 15 aa, Artificial Sequence
  Synthetic Polypeptide
EAAAKEAAAKEAAAK

SEQ ID NO: 16 - protein, 20 aa, Artificial Sequence
  Synthetic Polypeptide
EAAAREAAAREAAAREAAAR

SEQ ID NO: 17 - protein, 17 aa, Artificial Sequence
  Synthetic Polypeptide
GGGGSGGGGSGGGGSAS

SEQ ID NO: 18 - protein, 9 aa, Artificial Sequence
  Synthetic Polypeptide
GGGGAGGGG

SEQ ID NO: 19 - skipped

SEQ ID NO: 20 - protein, 6 aa, Artificial Sequence
  Synthetic Polypeptide
GSGSGS

SEQ ID NO: 21 - protein, 10 aa, Artificial Sequence
  Synthetic Polypeptide
GSGSGSGSGS

SEQ ID NO: 22 - protein, 7 aa, Artificial Sequence
  Synthetic Polypeptide
GGGGSAS

SEQ ID NO: 23 - protein, 20 aa, Artificial Sequence
  Synthetic Polypeptide
APAPAPAPAPAPAPAPAPAP

SEQ ID NO: 24 - protein, 4 aa, Artificial Sequence
  Synthetic Polypeptide
CPPC

SEQ ID NO: 25 - protein, 5 aa, Artificial Sequence
  Synthetic Polypeptide
GGGGS

SEQ ID NO: 26 - protein, 10 aa, Artificial Sequence
  Synthetic Polypeptide
GGGGSGGGGS

SEQ ID NO: 27 - protein, 15 aa, Artificial Sequence
  Synthetic Polypeptide
GGGGSGGGGSGGGGS

SEQ ID NO: 28 - protein, 20 aa, Artificial Sequence
  Synthetic Polypeptide
GGGGSGGGGSGGGGSGGGGS

SEQ ID NO: 29 - protein, 25 aa, Artificial Sequence
  Synthetic Polypeptide
GGGGSGGGGSGGGGSGGGGSGGGGS

SEQ ID NO: 30 - protein, 30 aa, Artificial Sequence
  Synthetic Polypeptide
GGGGSGGGGSGGGGSGGGGSGGGGSGGGGS

SEQ ID NO: 31 - protein, 35 aa, Artificial Sequence
  Synthetic Polypeptide
GGGGSGGGGSGGGGSGGGGSGGGGSGGGGSGGGGS

SEQ ID NO: 32 - protein, 40 aa, Artificial Sequence
  Synthetic Polypeptide
GGGGSGGGGSGGGGSGGGGSGGGGSGGGGSGGGGSGGGGS

SEQ ID NO: 33 - protein, 16 aa, Artificial Sequence
  Synthetic Polypeptide
GGSGGSGGGGSGGGGS

SEQ ID NO: 34 - protein, 8 aa, Artificial Sequence
  Synthetic Polypeptide
GGGGGGGG

SEQ ID NO: 35 - protein, 6 aa, Artificial Sequence
  Synthetic Polypeptide
GGGGGG

SEQ ID NO: 36 - protein, 5 aa, Artificial Sequence
  Synthetic Polypeptide
EAAAK

SEQ ID NO: 37 - protein, 10 aa, Artificial Sequence
  Synthetic Polypeptide
EAAAKEAAAK

SEQ ID NO: 38 - protein, 15 aa, Artificial Sequence
  Synthetic Polypeptide
EAAAKEAAAKEAAAK

SEQ ID NO: 39 - protein, 12 aa, Artificial Sequence
  Synthetic Polypeptide
AEAAAKEAAAKA

SEQ ID NO: 40 - protein, 17 aa, Artificial Sequence
  Synthetic Polypeptide
AEAAAKEAAAKEAAAKA

SEQ ID NO: 41 - protein, 22 aa, Artificial Sequence
  Synthetic Polypeptide
AEAAAKEAAAKEAAAKEAAAKA

SEQ ID NO: 42 - protein, 27 aa, Artificial Sequence
  Synthetic Polypeptide
AEAAAKEAAAKEAAAKEAAAKEAAAKA

SEQ ID NO: 43 - protein, 46 aa, Artificial Sequence
  Synthetic Polypeptide
AEAAAKEAAAKEAAAKEAAAKALEAEAAAKEAAAKEAAAKEAAAKA

SEQ ID NO: 44 - protein, 5 aa, Artificial Sequence
  Synthetic Polypeptide
PAPAP

SEQ ID NO: 45 - protein, 18 aa, Artificial Sequence
  Synthetic Polypeptide
KESGSVSSEQLAQFRSLD

SEQ ID NO: 46 - protein, 12 aa, Artificial Sequence
  Synthetic Polypeptide
GSAGSAAGSGEF

SEQ ID NO: 47 - protein, 5 aa, Artificial Sequence
  Synthetic Polypeptide
GGGSE

SEQ ID NO: 48 - protein, 5 aa, Artificial Sequence
  Synthetic Polypeptide
GSESG

SEQ ID NO: 49 - protein, 5 aa, Artificial Sequence
  Synthetic Polypeptide
GSEGS

SEQ ID NO: 50 - protein, 35 aa, Artificial Sequence
  Synthetic Polypeptide
GEGGSGEGSSGEGSSSEGGGSEGGGSEGGGSEGGS

SEQ ID NO: 51 - protein, 234 aa, Artificial Sequence
  Synthetic Polypeptide
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic Polypeptide
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 143 aa, Artificial Sequence
  Synthetic polymer.
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 58 - protein, 133 aa, Artificial Sequence
  Synthetic polymer.
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 59 - protein, 510 aa, Artificial Sequence
  Synthetic polymer.
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 60 - protein, 133 aa, Artificial Sequence; identical to SEQ ID NO: 58
  Synthetic polymer.

SEQ ID NO: 61 - protein, 583 aa, Artificial Sequence
  Synthetic polymer.
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 62 - protein, 498 aa, Artificial Sequence
  Synthetic polymer.
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTP

SEQ ID NO: 63 - protein, 215 aa, Artificial Sequence
  Synthetic polymer.
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 64 - protein, 947 aa, Artificial Sequence
  Synthetic polymer.
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

(19) United States
(12) Patent Application Publication
(10) Pub. No.: US 2022/0259281 A1
(43) Pub. Date: Aug. 18, 2022

(54) CHIMERIC PROTEIN EXPRESSING T-CELLS

(71) Applicant: Shattuck Labs, Inc., Austin, TX (US)

(72) Inventors: Taylor SCHREIBER, Austin, TX (US); George FROMM, Austin, TX (US); Suresh DE SILVA, Austin, TX (US)

(21) Appl. No.: 17/618,604

(22) PCT Filed: Jun. 22, 2020
(86) PCT No.: PCT/US20/38932

§ 371 Date: Dec. 13, 2021

Related U.S. Application Data
(60) Provisional application No. 62/864,791, filed on Jun. 21, 2019.

(51) Int. Cl. C07K 14/725; A61P 37/06; C12N 15/90; C07K 16/24

(52) U.S. Cl. CPC C07K 14/7051; A61P 37/06; C12N 15/90; C07K 16/24; C07K 2317/55; C07K 2317/24; C07K 2317/524; C07K 2317/526; C07K 2319/30

(57) ABSTRACT

The present invention relates to, inter alia, compositions and methods, including engineered T cells that express chimeric antigen receptors and heterologous chimeric proteins that find use in the treatment of cancer.

### PRIORITY

This application claims the benefit of, and priority to, U.S. Application No. 62/864,791, filed Jun. 21, 2019, which is hereby incorporated by reference in its entirety.

### TECHNICAL FIELD

The present invention relates to, inter alia, compositions and methods, including engineered T cells that express chimeric antigen receptors and heterologous chimeric proteins that find use in the treatment of cancer.

### DESCRIPTION OF THE TEXT FILE SUBMITTED ELECTRONICALLY

This application contains a sequence listing. It has been submitted electronically via EFS-Web as an ASCII text file entitled “SHK-021PC_SequenceListing_ST25”. The sequence listing is 57,782 bytes in size, and was prepared on or about Jun. 17, 2020. The sequence listing is hereby incorporated by reference in its entirety.

### BACKGROUND

Cancer is a broad group of diseases involving deregulated cell growth in which cancer cells divide and grow uncontrollably. Among the over two-hundred known cancer types that affect humans, safe and effective treatment options are available for only a subset of cancer types. Engineered T cell based immunotherapies, including CAR-T cells, have been successfully used for a few cancer types. However, the engineered T cells do not remain active in vivo for long periods of time and/or the anti-cancer activity of the engineered T cells is relatively low. There remains unmet needs for compositions and methods with improved anti-cancer activity.

### SUMMARY

In various aspects, the present invention provides compositions and methods that are useful in cancer immunotherapies. For instance, the present invention, in part, relates to an engineered T cell that expresses a chimeric antigen receptor and expresses a heterologous chimeric protein. The chimeric antigen receptor and the heterologous chimeric protein are each capable of forming a synapse between a cancer cell and the engineered T cell, thereby allowing for improved anti-cancer targeting and functioning. The present compositions and methods overcome various deficiencies in the field of cancer.

An aspect of the present invention is an engineered T cell that expresses a chimeric antigen receptor and a heterologous chimeric protein. The chimeric antigen receptor comprises an antigen-binding domain, a transmembrane domain, and an intracellular domain, which comprises a costimulatory domain and/or a signaling domain. The heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

An aspect of the present invention is an engineered T cell that expresses a heterologous chimeric protein, wherein the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

In an aspect, the present invention provides a method for manufacturing an engineered T cell. The method comprises steps of: (i) obtaining a T-cell or T-cell progenitor cell from a subject; (ii) transfecting the T-cell or T-cell progenitor cell with a first DNA molecule encoding a chimeric antigen receptor and designed to be integrated into the genome of the T cell or T-cell progenitor; and (iii) transfecting the T-cell or T-cell progenitor cell with a second DNA molecule encoding a heterologous chimeric protein and designed to be integrated into the genome of the T cell or T-cell progenitor. The heterologous chimeric protein of this aspect comprises the general structure: N terminus-(a)-(b)-(c)-C terminus, in which (a) is a first domain comprising an extracellular domain of a first transmembrane protein, (c) is a linker domain adjoining the first and second domains, and (b) is a second domain comprising an extracellular domain of a second transmembrane protein. In embodiments, step (ii) precedes step (iii). In embodiments, step (iii) precedes step (ii). In embodiments, step (ii) and step (iii) are contemporaneous.

In another aspect, the present invention provides another method for manufacturing an engineered T cell. The method comprising steps of: (i) obtaining a T-cell or T-cell progenitor cell that has been transfected with a first DNA molecule encoding a chimeric antigen receptor and designed to be integrated into the genome of the T cell or T-cell progenitor; and (ii) transfecting the T cell or T-cell progenitor cell of step (i) with a second DNA molecule encoding a heterologous chimeric protein and designed to be integrated into the genome of the T cell or T-cell progenitor. In this aspect, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

In yet another aspect, the present invention provides yet another method for manufacturing an engineered T cell. The method comprises steps of: (i) obtaining a T-cell or T-cell progenitor cell that has been transfected with a first DNA molecule encoding a heterologous chimeric protein and designed to be integrated into the genome of the T cell or T-cell progenitor; and (ii) transfecting the T cell or T-cell progenitor cell of step (i) with a second DNA molecule encoding a chimeric antigen receptor and designed to be integrated into the genome of the T cell or T-cell progenitor. In this aspect, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

In yet another aspect, the present invention provides yet another method for manufacturing an engineered T cell. The method comprises steps of: (i) obtaining a T-cell or T-cell progenitor cell, and (ii) transfecting the T cell or T-cell progenitor cell of step (i) with a DNA molecule encoding a chimeric antigen receptor and designed to be integrated into the genome of the T cell or T-cell progenitor. In this aspect, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

In yet another aspect, the present invention provides a method of analyzing an engineered T cell. The method comprises steps of: (A) manufacturing the engineered T cell, (B) culturing the engineered T cell, (C) isolating culture supernatant of the engineered T cell, optionally enriching or partially purifying the culture supernatant, (D) contacting the culture supernatant of the engineered T cell with a second cell, and (E) analyzing expression of a cytokine or chemokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A, ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In yet another aspect, the present invention provides a method of analyzing an engineered T cell. The method comprises steps of: (A) manufacturing the engineered T cell, (B) co-culturing the engineered T cell with a second cell, and (C) analyzing expression of a cytokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

An aspect of the present invention is a method for obtaining a population of engineered T cells. The method comprises obtaining an engineered T cell manufactured by a herein-disclosed method and culturing the engineered T cell in a medium that enhances proliferation of the engineered T cell, thereby obtaining a population of engineered T cells.

Other aspects of the present invention include a composition comprising a herein-disclosed engineered T cell and a pharmaceutical composition comprising the composition and a pharmaceutically acceptable excipient.

Another aspect is a herein-disclosed engineered T cell for use as a medicament in the treatment of a cancer.

Yet another aspect is the use of a herein-disclosed engineered T cell in the manufacture of a medicament.

In another aspect, the present invention provides a population of engineered T cells obtained by a herein-disclosed method.

Another aspect is a pharmaceutical composition comprising a herein-disclosed population of engineered T cells and a pharmaceutically acceptable excipient.

In another aspect, the present invention provides a method for treating a cancer in a subject in need thereof. The method comprises administering to the subject a therapeutically effective amount of a herein-disclosed pharmaceutical composition.

Any aspect or embodiment disclosed herein can be combined with any other aspect or embodiment as disclosed herein.

### BRIEF DESCRIPTION OF THE DRAWINGS

FIG. 1 shows a non-limiting schematic illustration of an engineered T cell that expresses a chimeric antigen receptor and a heterologous chimeric protein and their interactions with cancer cells.

FIG. 2A to FIG. 2D show schematic illustrations of Type I transmembrane proteins (FIG. 2A and FIG. 2B, left proteins) and Type II transmembrane proteins (FIG. 2A and FIG. 2B, right proteins). A Type I transmembrane protein and a Type II transmembrane protein may be engineered such that their transmembrane and intracellular domains are omitted and the transmembrane proteins' extracellular domains are adjoined using a linker domain to generate a single chimeric protein. As shown in FIG. 2C and FIG. 2D, the extracellular domain of a Type I transmembrane protein and the extracellular domain of a Type II transmembrane protein combined into a single heterologous chimeric protein.

FIG. 3 shows an ELISA assay demonstrating the expression and secretion of the PD-1-Fc-OX40L heterologous chimeric protein from stably transfected Jurkat T cells.

FIG. 4 shows an ELISA assay demonstrating the expression and secretion of the CSF1R-Fc-CD40L heterologous chimeric protein from stably transfected Jurkat T cells.

FIG. 5A to FIG. 5C demonstrate the presence of the type I extracellular domain (ECD) and type II ECDs of the human PD-1-Fc-4-1BBL (FIG. 5A), PD-1-Fc-OX40L (FIG. 5B), and CSF1R-Fc-CD40L (FIG. 5C) heterologous chimeric proteins secreted by Jurkat cells. Jurkat parental cells, Jurkat cells secreting the hPD-1-Fc-4-1BBL chimeric protein (Jurkat/hPD-1-Fc-4-1BBL cells), Jurkat cells secreting the hPD-1-Fc-OX40L chimeric protein (Jurkat/hPD-1-Fc-OX40L cells), or Jurkat cells secreting the hCSF1R-Fc-CD40L chimeric protein (Jurkat/hCSF1R-Fc-CD40L cells) were cultured. “Nest culture supernatant” is the concentrated and de-salted culture supernatant prepared as described in Example 2. Dotted lines shows background level of MSD signal. For FIG. 5A, the human PD-1-Fc-4-1BBL protein was used as a positive control. An anti-human PD-1 antibody was coated on a plate. Indicated amounts of the hPD-1-Fc-4-1BBL protein or the indicated dilutions neat culture supernatants of Jurkat parental cells or Jurkat/hPD-1-Fc-4-1BBL cells were added to the plate. The plate was incubated to allow capture of any protein by the plate-bound anti-human PD-1 antibody. The anti-human PD-1 antibody-bound chimeric protein was detected using an anti-human 4-1BBL antibody using the MESO SCALE DISCOVERY (MSD) platform. For FIG. 5B, the human PD-1-Fc-OX40L protein was used as a positive control. An anti-human PD-1 antibody was coated on a plate. Indicated amounts of the hPD-1-Fc-OX40L protein, or the indicated dilutions neat culture supernatants of Jurkat parental cells or Jurkat/hPD-1-Fc-OX40L cells were added to the plate. The plate was incubated to allow capture of any protein by the plate-bound anti-human PD-1 antibody. The anti-human PD-1 antibody-bound chimeric protein was detected using an anti-human OX40L antibody using the MSD platform. For FIG. 5C, the human CSF1R-Fc-CD40L protein was used as a positive control. An anti-human CSF1R antibody was coated on a plate. Indicated amounts of the hCSF1R-Fc-CD40L protein, or the indicated dilutions neat culture supernatants of Jurkat parental cells or Jurkat/hCSF1R-Fc-CD40L cells were added. The plate was incubated to allow capture of any protein by the plate-bound anti-human CSF1R antibody. The anti-human CSF1R antibody-bound chimeric protein was detected using an anti-human CD40L antibody using the MSD platform.

FIG. 6A to FIG. 6C show the flow cytometry profiles of CHO-K1 cells that were engineered to over-express human CD40 (the CHO-K1/hCD40 cells), PD-L1 (the CHO-K1/hPD-L1 cells), or OX40 (the CHO-K1/hOX40 cells) stained with anti-CD40, anti-PD-L1 and anti-OX40 antibodies, respectively, in comparison with CHO-K1 parental cells. FIG. 6A shows the over-expression of human CD40 in CHO-K1/hCD40 cells compared to CHO-K1 parental cells. FIG. 6B shows the over-expression of human PD-L1 in CHO-K1/hPD-L1 cells compared to CHO-K1 parental cells. FIG. 6C shows the over-expression of human OX40 in CHO-K1/hOX40 cells compared to CHO-K1 parental cells.

FIG. 7A and FIG. 7B show the expression of human 4-1BB (FIG. 7A) and OX40 (FIG. 7B) receptors on HT1080/cells over-expressing human 4-1BB (the HT1080/h4-1BB cells) as determined by flow cytometry.

FIG. 8A and FIG. 8B show the quantitation of the binding of the ECDs of human PD-1 and 4-1BBL present in the hPD-1-Fc-4-1BBL chimeric protein produced by the Jurkat/hPD-1-Fc-4-1BBL cells to cells expressing 4-1BB or PD-L1 as determined by flow cytometry. Dotted lines shows background signal. FIG. 8A shows the binding to HT1080 cells overexpressing human 4-1BB (HT1080/h4-1BB cells) by the chimeric protein in neat culture supernatant of Jurkat parental cells (black bars) or Jurkat/hPD-1-Fc-4-1BBL cells (grey bars). FIG. 8B shows the binding to CHO-K1 cells overexpressing human PD-L1 by the chimeric protein in neat culture supernatant of Jurkat parental cells (black bars) or Jurkat/hPD-1-Fc-4-1BBL (grey bars).

FIG. 9A and FIG. 9B show the quantitation of the binding of the ECDs of human PD-1 and OX40L present in the hPD-1-Fc-OX40L chimeric protein produced by the Jurkat/hPD-1-Fc-OX40L cells to cells expressing OX40 or PD-L1 as determined by flow cytometry. Dotted lines shows background signal. FIG. 9A shows the binding to the CHO-K1/hPD-L1 cells by the chimeric protein in neat culture supernatant of Jurkat parental cells (black bars) or Jurkat/hPD-1-Fc-OX40L cells (grey bars). FIG. 9B shows the binding to CHO-K1 cells overexpressing human OX40 by the chimeric protein in neat culture supernatant of Jurkat parental cells (black bars) or Jurkat/hPD-1-Fc-OX40L (grey bars).

FIG. 10A and FIG. 10B show the quantitation of the binding of the ECDs of human CSF1R and CD40L present in the hPD-1-Fc-OX40L chimeric protein produced by the Jurkat/hPD-1-Fc-OX40L cells to their respective ligands. Dotted lines shows background signal. FIG. 10A shows the quantitation of the binding to the CHO-K1/hCD40 cells by the chimeric protein in neat culture supernatant of Jurkat parental cells (black bars) or Jurkat/hCSF1R-Fc-CD40L cells (grey bars) as measured by flow cytometry. FIG. 10B shows the binding of the hCSF1R-Fc-CD40L chimeric protein in neat culture supernatant of Jurkat parental cells (black bars) or Jurkat/hCSF1R-Fc-CD40L cells to human CSF1 protein as detected using the MSD platform. Human recombinant CSF1-coated MSD plates were incubated with increasing amounts of the culture supernatant of the Jurkat parental or Jurkat/hCSF1R-Fc-CD40L cells produced as discussed in Example 2. Binding was detected using anti-human CD40L-biotin, followed by the MSD streptavidin reagent.

FIG. 11A to FIG. 11C show the induction of cytokine genes by the chimeric hPD-1-Fc-4-1BBL protein produced by the Jurkat/hPD-1-Fc-4-1BBL cells. 250,000 HT1080/h4-1BB cells were cultured. The indicated amounts of the neat culture supernatant of Jurkat parental cells or Jurkat/hPD-1-Fc-4-1BBL cells were added to the HT1080/h4-1BB cell culture. After 3 hours, RNA was isolated from HT1080/h4-1BB cells, cDNA was synthesized, and gene expression of ACTB (FIG. 11A), CXCL8 (FIG. 11B), and CCL2 (FIG. 11C) was assessed by qRT-PCR. GAPDH was used to normalize gene expression and fold-induction according to the delta-delta CT method. Dotted lines shows background signal from the HT1080/h4-1BB cells treated with the neat culture supernatant of Jurkat parental cells.

FIG. 12 shows the induction of IL-8 by the chimeric hPD-1-Fc-4-1BBL protein produced by the Jurkat/hPD-1-Fc-4-1BBL cells. 250,000 HT1080/h4-1BB cells were cultured. Neat culture supernatants of Jurkat parental cells or Jurkat/hPD-1-Fc-4-1BBL cells were added to 25% to the HT1080/h4-1BB cell culture. After 3 hours, media were removed from the HT1080/h4-1BB cell culture and IL-8 was assessed by ELISA. Dotted lines shows background signal from the HT1080/h4-1BB cells treated with the neat culture supernatant of Jurkat parental cells.

FIG. 13A and FIG. 13B show the induction of CCL2 expression by the chimeric hPD-1-Fc-OX40L protein produced by the Jurkat/hPD-1-Fc-OX40L cells. 250,000 HT1080/h4-1BB cells were cultured. The indicated amounts of the neat culture supernatant of Jurkat parental cells or Jurkat/hPD-1-Fc-OX40L cells were added to the HT1080/h4-1BB cell culture. After 3 hours, RNA was isolated from HT1080/h4-1BB cells, cDNA was synthesized, and gene expression of ACTB (housekeeping control gene; FIG. 13A) and CCL2 (FIG. 13B) was assessed by qRT-PCR. GAPDH was used to normalize gene expression and fold-induction according to the delta-delta CT method. Dotted lines shows background signal from the HT1080/h4-1BB cells treated with the neat culture supernatant of Jurkat parental cells.

FIG. 14A and FIG. 14B show the induction of signaling in the PATHHUNTER U2OS-NIK/NFκB reporter cells (DISCOVERX) by the Jurkat/hCSF1R-Fc-CD40L cells. Dotted lines shows background signal. FIG. 14A demonstrates that the PATHHUNTER U2OS-NIK/NFκB reporter cells express human CD40 (hCD40). The PATHHUNTER U2OS-NIK/NFκB reporter cells were stained using APC-conjugated isotype antibody or an anti-human CD40-APC antibody, and analyzed by flow cytometry. FIG. 14B shows the induction of NFκB signaling in the PATHHUNTER U2OS-NIK/NFκB reporter cells by the Jurkat/hCSF1R-Fc-CD40L cells. The indicated numbers of Jurkat parental cells or Jurkat/hCSF1R-Fc-CD40L cells were co-cultured with the PATHHUNTER U2OS-NIK/NFκB reporter cells. 3-fold serial dilutions of 15 μg/mL purified CSF1R-Fc-CD40L chimeric protein were used as a positive controls. After 6 hours in culture, the DISCOVERX luminescence reagent was added, and bioluminescence was determined using a PROMEGA GLOMAX instrument.

FIG. 15 shows the induction of IL-8 secretion by the Jurkat/hPD1-Fc-4-1BBL cells when co-cultured with cells expressing 4-1BB. 250,000 HT1080/4-1BB cells were cultured overnight. The next day either Jurkat parental cells or the Jurkat/hPD1-Fc-4-1BBL cells were added to the HT1080/4-1BB cells at the indicated ratio. After 6 hours of co-culture, the supernatant was removed and IL-8 was assessed by ELISA. Dotted lines shows background signal from the untreated HT1080/h4-1BB cells.

### DETAILED DESCRIPTION

The present invention is based, in part, on the creation of an engineered T cell that expresses a chimeric antigen receptor and expresses a heterologous chimeric protein.

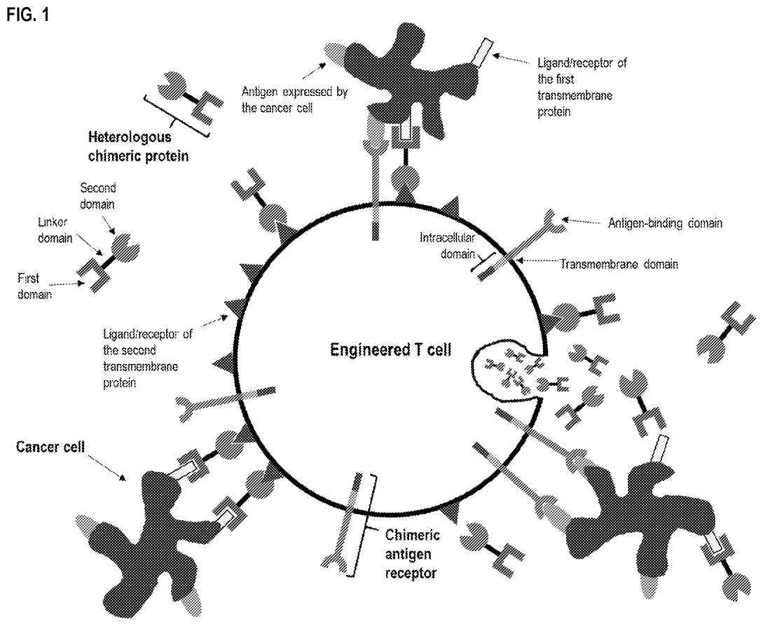

The present invention and advantages thereof are illustrated in FIG. 1. Shown is the engineered T cell that expresses chimeric antigen receptors and expresses heterologous chimeric proteins. The chimeric antigen receptors, comprising at least an antigen-binding domain, a transmembrane domain, and an intracellular domain, are transmembrane proteins in which their antigen-binding domains are located in the extracellular space. There, the antigen-binding domain is able to bind to antigens presented on the surface of cancer cells. When the antigen-binding domain is bound to a cancer cell, a stable synapse is formed between the engineered T cell and the cancer cell; this stable synapse favors an anti-cancer effect/tumor reduction by the engineered T cell (which becomes activated as an antigen-bound signal transmits to the cell via the chimeric antigen receptor's intracellular domain). As shown in FIG. 1, some cancer cells are bound to the engineered T cell via a chimeric antigen receptor and some cancer cells are additionally/alternatively bound to the engineered T cell via a heterologous chimeric protein. The heterologous chimeric proteins, comprising at least a first domain (including a portion of a first transmembrane protein which binds a ligand/receptor on a cancer cell), a linker domain, and a second domain (including a portion of a second transmembrane protein which binds a ligand/receptor on a T cell), are expressed and secreted by the engineered T cell. When the first domain is bound to a cancer cell and when the second domain is bound to the engineered T cell, a stable synapse is also formed between the engineered T cell and the cancer cell; this stable synapse also favors an anti-cancer effect/tumor reduction by the engineered T cell (which is activated in response to binding of the second domain to engineered T cell) and blocks an inhibitory signal (which is transmitted by the first transmembrane protein's ligand/receptor presented by the cancer cell). As shown, stable synapses can form comprising only chimeric antigen receptors; stable synapses can form comprising only heterologous chimeric proteins; or can form synergistic stable synapses with both chimeric antigen receptors and heterologous chimeric proteins. Since, the chimeric antigen receptor and the heterologous chimeric protein are each capable of forming a synapse between a cancer cell and the engineered T cell, this allows for an improvement in anti-cancer targeting and function. Thus, the engineered T cells of the present invention, compositions comprising the same, and treatment methods using these provide superior cancer cell killing and tumor reduction and overcome various deficiencies in the field of cancer therapeutics.

Chimeric Antigen Receptor (CAR)

The present invention provides, inter alia, an engineered T cell that expresses a chimeric antigen receptor (CAR) and a heterologous chimeric protein. The chimeric antigen receptor comprises an antigen-binding domain, a transmembrane domain, and an intracellular domain, which comprises a costimulatory domain and/or a signaling domain.

Chimeric antigen receptors (CARs, also known as chimeric immunoreceptors, chimeric T cell receptors or artificial T cell receptors) are receptor proteins that have been engineered to give T cells the new ability to target a specific protein. The receptors are chimeric because they combine both antigen-binding and T-cell activating functions into a single receptor.

There are several generations of chimeric antigen receptors. For example, “First generation” chimeric antigen receptors typically comprise an extracellular antigen-binding domain (e.g., a single-chain variable fragments (scFv)) fused to a transmembrane domain, which is fused to cytoplasmic/intracellular signaling domain of the T cell receptor (TCR) chain. “First generation” chimeric antigen receptors typically have the intracellular signaling domain from the CD3ζ-chain, which is the primary transmitter of signals from endogenous TCRs. “First generation” chimeric antigen receptors can provide de novo antigen recognition and cause activation of both CD4+ and CD8+ T cells through their CD3ζ-chain signaling domain in a single fusion molecule, independent of HLA-mediated antigen presentation, as required by native TCRs. “Second generation” chimeric antigen receptors add intracellular signaling domains from various co-stimulatory molecules (e.g., CD28, 4-1BB, ICOS, and OX40) to the cytoplasmic tail of the chimeric antigen receptor to provide additional signals to the T cell. “Second generation” chimeric antigen receptors comprise both co-stimulation (e.g., CD28 or 4-1BB) and activation (CD3ζ) domains. Preclinical studies have indicated that “Second Generation” chimeric antigen receptors can improve the anti-cancer activity of T cells. For example, robust efficacy of “Second Generation” chimeric antigen receptor-engineered T cells have been used in clinical trials targeting the CD19 molecule in patients with chronic lymphoblastic leukemia (CLL) and acute lymphoblastic leukemia (ALL). “Third generation” chimeric antigen receptors comprise multiple co-stimulation (e.g., CD28 and 4-1BB) and activation (CD3ζ) domains. “Fourth Generation” chimeric antigen receptors comprise pro-inflammatory cytokines or co-stimulatory ligands co-expressed by the chimeric antigen receptor-engineered T cells. A herein-disclosed chimeric antigen receptor can include any generation of chimeric antigen receptor, as described above.

In embodiments, there is provided an engineered T cell that comprises a first DNA segment encoding an antigen-binding domain, e.g. single-chain Fv domain (scFv), comprising a VL linked to a VH of a specific antibody by a flexible linker and a second DNA segment encoding partially or entirely the transmembrane and cytoplasmic domains of an endogenous protein, where endogenous protein is expressed on the surface of lymphocytes and triggers the activation and/or proliferation of said lymphocytes, and a third DNA segment encoding any of the heterologous chimeric proteins described herein. In embodiments, upon transfection to lymphocytes, the lymphocytes express both the scFv domain and the domains of said endogenous protein in one single, continuous chain on the surface of the transfected lymphocytes. In embodiments, the transfected lymphocytes are triggered to activate and/or proliferate and have MHC non-restricted antibody-type specificity when the expressed scFv domain binds to its antigen. In embodiments, the endogenous protein is a lymphocyte receptor chain, a polypeptide of the TCR/CD3 complex, or a subunit of the Fc or IL-2 receptor.

In embodiments, the engineered T cell is engineered to allow for allogeneic uses (e.g. in the method of treatment described herein). In embodiments, the engineered T cell is modified to functionally impair or to reduce expression of the endogenous T cell receptor (TCR), and/or modified to express at least one functional exogenous non-TCR that optionally comprises a chimeric receptor comprising a NKG2D ligand binding domain attached to a CD3 £ signaling domain. In embodiments, the engineered T cell is suitable for use in human therapy, e.g. the engineered T cell elicits no or a reduced GVHD response in a histoincompatible human recipient as compared to the GVHD response elicited by a primary human T cell isolated from the same human donor that is not modified or only modified to express at least one functional exogenous non-TCR.

Antigen-Binding Domain of a Chimeric Antigen Receptor

A herein-disclosed chimeric antigen receptor comprises an antigen-binding domain that is capable of binding an antigen expressed by a cancer cell.

In embodiments, the antigen-binding domain of a chimeric antigen receptor useful in the present invention specifically binds to a cancer cell-specific antigen, e.g., a human cancer cell-specific antigen.

In embodiments, the antigen-binding domain is an antibody, an antibody fragment, an scFv, a Fv, a Fab, a (Fab′)2, a single domain antibody (SDAB), a VH or VL domain, or a camelid VHH domain, e.g., a human scFv, human Fv, human Fab, human (Fab′)2, human single domain antibody (SDAB), or human VH or VL domain or a humanized scFv, humanized Fv, humanized Fab, humanized (Fab′)2, humanized single domain antibody (SDAB), or humanized VH or VL domain.

In embodiments, the antigen expressed by the cancer cell (and recognized by the antigen-binding domain) is selected from the group consisting of B cell maturation antigen (BCMA), CD10, CD19, CD20, CD22, CD30, CD33, CD34, CD38, CD56, CD80/86, CD117, CD123, CD123, CD133, CD138, carcinoembryonic antigen (CEA), CS1/CD319/SLAMF7, disialoganglioside (GD2), EphA2, epidermal growth factor receptor (EGFR), epithelial cell adhesion molecule (EpCAM), glypican-3 (GPC3), HER2, integrin β7, Lewis-Y, mesothelin, MUC1, PD-L1, and Prostate Stem Cell Antigen (PSCA).

Optionally, the chimeric antigen receptor comprise a spacer between the antigen-binding domain and the engineered T cell's outer membrane. An ideal spacer enhances the flexibility of the antigen-binding domain relative to the rest of the receptor, thereby reducing the spatial constraints between the receptor and its target antigen. This flexibility promotes antigen binding and synapse formation between the engineered T cells and the cancer cells. A spacer may comprise a hinge domain from an IgG or from CD8.

Binding of the cancer cell antigen by the antigen-binding domain is a first step in forming a stable synapse between the engineered T cell and the cancer cell.

Transmembrane Domain of a Chimeric Antigen Receptor

In embodiments, the transmembrane domain of a chimeric antigen receptor useful in the present invention comprises a transmembrane domain of a protein selected from the group consisting of the alpha chain of the T-cell receptor, the beta chain of the T-cell receptor, or the zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDIIa, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDI Ib, ITGAX, CDIIc, ITGBI, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a combination thereof.

In embodiments, the transmembrane domain of a chimeric antigen receptor useful in the present invention comprises a hydrophobic alpha helix that spans at least a portion of the membrane. The transmembrane domain is essential for the stability of the receptor as a whole. Different transmembrane domains result in different receptor stability. The CD28 transmembrane domain is known to result in a highly expressed, stable receptor.

After a stable synapse has formed between the engineered T cell and the cancer cell, nearby chimeric antigen receptors cluster.

Intracellular Domain of a Chimeric Antigen Receptor

In embodiments, the intracellular domain of a chimeric antigen receptor useful in the present invention comprises a costimulatory domain comprising a portion of the intracellular domain of CD27, CD28, 4-IBB, OX40, CD30, CD40, PD-1, ICOS, lymphocyte function-associated antigen-1 (LFA-1), CD2, CD7, LIGHT, NKG2C, B7-H3, or a ligand that specifically binds CD83, or a combination thereof.

In embodiments, the intracellular domain of a chimeric antigen receptor useful in the present invention comprises a costimulatory domain from CD28, OX40, and/or 4-1BB.

In embodiments, the intracellular domain of a chimeric antigen receptor useful in the present invention comprises a signaling domain comprising a portion of the cytoplasmic domain of CD3-ζ. Normal T cell receptor activation relies on the phosphorylation of immunoreceptor tyrosine-based activation motifs (ITAMs) present in the cytoplasmic domain of CD3-ζ. To mimic this process, a signaling domain comprising CD3-ζ is able to transmit an activation signal to the engineered T cell once an antigen is bound by the chimeric antigen receptor's antigen binding domain.

In embodiments, the intracellular domain of a chimeric antigen receptor useful in the present invention comprises a signaling domain from CD3-ζ.

In embodiments, the intracellular domain of a chimeric antigen receptor useful in the present invention comprises both a costimulatory domain and a signaling domain.

After a stable synapse has formed between the engineered T cell and the cancer cell and nearby chimeric antigen receptors have clustered, a signal is transmitted to the cell via its intracellular domain.

An aspect of the present invention is an engineered T cell that expresses a heterologous chimeric protein, wherein the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein. In embodiments, the T cell is a T-cell progenitor cell. In embodiments, the T-cell or T-cell progenitor cell is obtained from a patient. In embodiments, the T-cell or T-cell progenitor cell is specific to a cancer antigen.

Heterologous Chimeric Protein

An aspect of the present invention is an engineered T cell that expresses a chimeric antigen receptor and a heterologous chimeric protein. The heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

In embodiments, the first domain comprises substantially the entire extracellular domain of the first transmembrane protein and/or the second domain comprises substantially the entire extracellular domain of the second transmembrane protein.

In embodiments, the first domain is capable of binding a ligand/receptor of the first transmembrane protein and the second domain is capable of binding a ligand/receptor of the second transmembrane protein.

In embodiments, the first domain is capable of inhibiting an immunosuppressive signal when bound to its ligand/receptor and/or the second domain is capable of activating an immune stimulatory signal when bound to its ligand/receptor.

In embodiments, the ligand/receptor of the first transmembrane protein is expressed by a cancer cell and/or the ligand/receptor of the second transmembrane protein is expressed by the engineered T cell and/or a native T cell.

Single-pass transmembrane proteins relevant to the present invention comprise an extracellular domain, a transmembrane domain, and an intracellular domain. The extracellular domain of a transmembrane protein is responsible for interacting with a soluble receptor/ligand or membrane-bound receptor/ligand (i.e., the membrane of an adjacent cell). The trans-membrane domain is responsible for localizing the transmembrane protein to the plasma membrane. And, the intracellular domain is responsible for transmitting a signal to the interior of the cell in response to contact of the extracellular domain with its ligand/receptor.

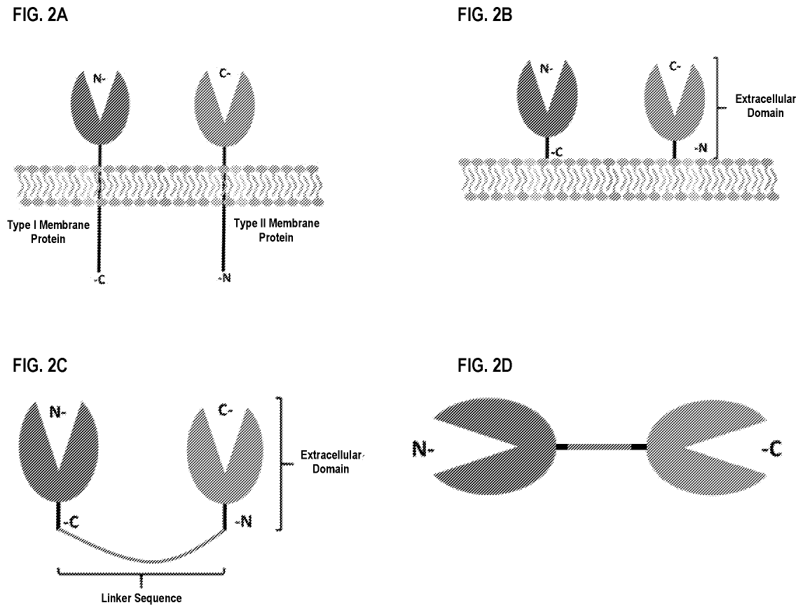

There are generally two types of single-pass transmembrane proteins: Type I transmembrane proteins, which have an extracellular amino terminus and an intracellular carboxy terminus (see, FIG. 2A and FIG. 2B, left proteins), and Type II transmembrane proteins, which have an extracellular carboxy terminus and an intracellular amino terminus (see, FIG. 2A and FIG. 2B, right proteins). Transmembrane proteins can be either receptors or ligands. For Type I transmembrane proteins, the amino terminus of the protein faces outside the cell, and therefore contains the functional domains that are responsible for interacting with other binding partners (either ligands or receptors) in the extracellular environment. For Type II transmembrane proteins, the carboxy terminus of the protein faces outside the cell, and therefore contains the functional domains that are responsible for interacting with other binding partners (either ligands or receptors) in the extracellular environment. Thus, these two types of transmembrane proteins have opposite orientations to each other relative to the cell membrane.

Heterologous chimeric proteins useful in the present invention comprise an extracellular domain of a first transmembrane protein and an extracellular domain of a second transmembrane protein which are connected directly or via a linker. As illustrated in FIG. 2C and FIG. 2D, when the domains are linked in an amino-terminal to carboxy-terminal orientation, the first domain is located on the “left’” side of the chimeric protein and is “outward facing” and the second domain is located on “right” side of the chimeric protein and is “outward facing”.

Importantly, since a heterologous chimeric protein useful in the present invention disrupts, blocks, reduces, inhibits, and/or sequesters the transmission of immunosuppressive signals and also transmits, provides, and/or activates immune stimulatory signals, it provides an anti-cancer effect by two distinct pathways; this dual-action is more likely to provide a therapeutic effect in a patient and/or to provide an enhanced therapeutic effect in a patient, e.g., when compared to therapeutics comprising two antibodies which bind to either of the ligands/receptors of the heterologous chimeric proteins or two fusion proteins each comprising a single ligand/receptor binding domain of the heterologous chimeric proteins), neither of which can create a stable synapse between a cancer cell and an engineered T cell. Additionally, since such heterologous chimeric proteins can act via two distinct pathways, they can be efficacious, at least, in patients who respond poorly to treatments that target one of the two pathways. Thus, a patient who is a poor responder to treatments acting via one of the two pathways can receive a therapeutic benefit by targeting the other pathway.

The heterologous chimeric proteins useful in the present invention are capable of forming a stable synapse between the cancer cell and the engineered T cell when its first domain is bound to the ligand/receptor of the first transmembrane protein expressed by the cancer cell and the second domain is bound to the ligand/receptor of the second transmembrane protein expressed by the engineered T cell. The stable synapse provides a spatial orientation that favors tumor reduction by the engineered T cell.

In a heterologous chimeric protein useful in the present invention, first transmembrane protein may be 2B4, 4-1BB, ACVR1b, ACVR2A, ACVR2B, AXL, B7-H3, BCMA, BTLA, BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8.

In embodiments, the first transmembrane protein is PD-1, CSF1R, TIM-3, BTLA, CTLA-4, LAG-3, B7-H3, TMIGD2, TIGIT, SIRPα, BTNL3, BTNL8, BTNL3A1, BTNL3A2, or VSIG8.

In a heterologous chimeric protein useful in the present invention, the second transmembrane protein may be 4-1BB ligand (4-1BBL), APRIL, BAFF, BTNL2, CD28, CD30 ligand (CD30L), CD40 ligand (CD40L), CD70, C-type lectin domain (CLEC) family members, FasL, GITR ligand (GITRL), LIGHT (CD258), LTa, LTa1b2, NKG2A, NKG2C, NKG2D, OX40 ligand (OX40L), RANKL, TL1A, TNFa, or TRAIL.

In embodiments, the second transmembrane protein is OX40L, CD40L, 4-1BBL, GITRL, LIGHT, CD70, CD30L, TRAIL, or TL1A.

In embodiments, a heterologous chimeric protein useful in the present invention comprises a variant of the extracellular domain of PD-1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of PD-1, e.g., human PD-1.

In embodiments, the extracellular domain of PD-1 has the following amino acid sequence:

(SEQ ID NO: 57)
LDSPDRPWNPPTFSPALLVVTEGDNATFTCSFSNTSESFVLNWYRMSPSNQ
TDKLAAFPEDRSQPGQDCRFRVTQLPNGRDFHMSVVRARRNDSGTYLCGAI
SLAPKAQIKESLRAELRVTERRAEVPTAHPSPSPRPAGQFQ.

In embodiments, a heterologous chimeric protein comprises a variant of the extracellular domain of PD-1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

In embodiments, the first domain of a heterologous chimeric protein comprises an amino acid sequence that is at least 95% identical to the amino acid sequence of SEQ ID NO: 57.

One of ordinary skill may select variants of the known amino acid sequence of PD-1 by consulting the literature, e.g. Zhang et al “Structural and Functional Analysis of the Costimulatory Receptor Programmed Death-1” Immunity. 2004 March; 20(3):337-47; Lin et al “The PD-1/PD-L1 complex resembles the antigen-binding Fv domains of antibodies and T cell receptors”, Proc Natl Acad Sci USA. 2008 Feb. 26; 105(8):3011-6; Zak et al “Structure of the Complex of Human Programmed Death 1, PD-1, and Its Ligand PD-L1, Structure. 2015 Dec. 1; 23(12):2341-2348; and Cheng et al “Structure and Interactions of the Human Programmed Cell Death 1 Receptor”, J Biol Chem. 2013 Apr. 26; 288(17):11771-85, each of which is incorporated by reference in its entirety.

In embodiments, a heterologous chimeric protein useful in the present invention comprises a variant of the extracellular domain of OX40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of OX40L, e.g., human OX40L.

In embodiments, the extracellular domain of OX40L has the following amino acid sequence:

(SEQ ID NO: 58)
QVSHRYPRIQSIKVQFTEYKKEKGFILTSQKEDEIMKVQNNSVIINCDGFY
LISLKGYFSQEVNISLHYQKDEEPLFQLKKVRSVNSLMVASLTYKDKVYLN
VTTDNTSLDDFHVNGGELILIHQNPGEFCVL.

In embodiments, a heterologous chimeric protein comprises a variant of the extracellular domain of OX40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a heterologous chimeric protein comprises an amino acid sequence that is at least 95% identical to the amino acid sequence of SEQ ID NO: 58.

One of ordinary skill may select variants of the known amino acid sequence of OX40L by consulting the literature, e.g. CROFT, et al., “The Significance of OX40 and OX40L to T cell Biology and Immune Disease,” Immunol Rev., 229(1), PP. 173-191, 2009 and BAUM, et al., “Molecular characterization of murine and human OX40/0X40 ligand systems: identification of a human OX40 ligand as the HTLV-1-regulated protein gp34,” The EMBO Journal, Vol. 13, No. 77, PP. 3992-4001, 1994, each of which is incorporated by reference in its entirety.

In embodiments, a heterologous chimeric protein useful in the present invention comprises: (a) a first domain comprising the amino acid sequence of SEQ ID NO: 57, (b) a second domain comprises the amino acid sequence of SEQ ID NO: 58, and (c) a linker comprises an amino acid sequence that is at least 95% identical to SEQ ID NO: 1, SEQ ID NO: 2, or SEQ ID NO: 3. Such a heterologous chimeric protein may be referred to as “PD-1-Fc-OX40L.”

In embodiments, a PD-1-Fc-OX40L chimeric protein used in the present invention and has the following amino acid sequence:

(SEQ ID NO: 59)
LDSPDRPWNPPTFSPALLVVTEGDNATFTCSFSNTSESFVLNWYRMSPSNQ
TDKLAAFPEDRSQPGQDCRFRVTQLPNGRDFHMSVVRARRNDSGTYLCGAI
SLAPKAQIKESLRAELRVTERRAEVPTAHPSPSPRPAGQFQSKYGPPCPPC
PAPEFLGGPSVFLFPPKPKDQLMISRTPEVTCVVVDVSQEDPEVQFNWYVD
GVEVHNAKTKPREEQFNSTYRVVSVLTVLHQDWLSGKEYKCKVSSKGLPSS
IEKTISNATGQPREPQVYTLPPSQEEMTKNQVSLTCLVKGFYPSDIAVEWE
SNGQPENNYKTTPPVLDSDGSFFLYSRLTVDKSRWQEGNVFSCSVLHEALH
NHYTQKSLSLSLGKIEGRMDQVSHRYPRIQSIKVQFTEYKKEKGFILTSQK
EDEIMKVQNNSVIINCDGFYLISLKGYFSQEVNISLHYQKDEEPLFQLKKV
RSVNSLMVASLTYKDKVYLNVTTDNTSLDDFHVNGGELILIHQNPGEFCV
L.

In embodiments, a heterologous chimeric protein useful in the present invention comprises a variant of the extracellular domain of 4-1BBL. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of 4-1BBL, e.g., human 4-1BBL.

In embodiments, the extracellular domain of 4-1BBL has the following amino acid sequence:

(SEQ ID NO: 60)
QVSHRYPRIQSIKVQFTEYKKEKGFILTSQKEDEIMKVQNNSVIINCDGFY
LISLKGYFSQEVNISLHYQKDEEPLFQLKKVRSVNSLMVASLTYKDKVYLN
VTTDNTSLDDFHVNGGELILIHQNPGEFCVL.

In embodiments, a heterologous chimeric protein comprises a variant of the extracellular domain of 4-1BBL. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the second domain of a heterologous chimeric protein comprises an amino acid sequence that is at least 95% identical to the amino acid sequence of SEQ ID NO: 60.

The 4-1BBL protein encoded is a cytokine that belongs to the tumor necrosis factor (TNF) ligand family. This transmembrane cytokine is a bidirectional signal transducer that acts as a ligand for TNFRSF9/4-1BB, which is a costimulatory receptor molecule in T lymphocytes. This cytokine and its receptor are involved in the antigen presentation process and in the generation of cytotoxic T cells. The receptor TNFRSF9/4-1BB is absent from resting T lymphocytes but rapidly expressed upon antigenic stimulation. The ligand encoded by this gene, TNFSF9/4-1BBL, has been shown to reactivate anergic T lymphocytes in addition to promoting T lymphocyte proliferation. This cytokine has also been shown to be required for the optimal CD8 responses in CD8 T cells. This cytokine is expressed in carcinoma cell lines, and is thought to be involved in T cell-cancer cell interaction.

One of ordinary skill may select variants of the known amino acid sequence of 4-1BBL by consulting the literature, e.g., Won et al., “The structure of the trimer of human 4-1BB ligand is unique among members of the tumor necrosis factor superfamily.” J. Biol. Chem. 285 (12), 9202-9210 (2010); Alderson et al., “Molecular and biological characterization of human 4-1BB and its ligand.” Eur. J. Immunol. 24 (9), 2219-2227 (1994); and Arch and Thompson “4-1BB and OX40 are members of a tumor necrosis factor (TNF)-nerve growth factor receptor subfamily that bind TNF receptor-associated factors and activate nuclear factor kappaB.” Mol. Cell. Biol. 18 (1), 558-565 (1998), each of which is incorporated by reference in its entirety.

In embodiments, a heterologous chimeric protein useful in the present invention comprises: (a) a first domain comprising the amino acid sequence of SEQ ID NO: 57, (b) a second domain comprises the amino acid sequence of SEQ ID NO: 60, and (c) a linker comprises an amino acid sequence that is at least 95% identical to SEQ ID NO: 1, SEQ ID NO: 2, or SEQ ID NO: 3. Such a heterologous chimeric protein may be referred to as “PD-1-Fc-4-1BBL”.

In embodiments, a PD-1-Fc-4-1BBL chimeric protein used in the present invention and has the following amino acid sequence:

(SEQ ID NO: 61)
LDSPDRPWNPPTFSPALLVVTEGDNATFTCSFSNTSESFVLNWYRMSPSNQ
TDKLAAFPEDRSQPGQDCRFRVTQLPNGRDFHMSVVRARRNDSGTYLCGAI
SLAPKAQIKESLRAELRVTERRAEVPTAHPSPSPRPAGQFQSKYGPPCPPC
PAPEFLGGPSVFLFPPKPKDQLMISRTPEVICVVVDVSQEDPEVQFNWYVD
GVEVHNAKTKPREEQFNSTYRVVSVLTVLHQDWLSGKEYKCKVSSKGLPSS
IEKTISNATGQPREPQVYTLPPSQEEMTKNQVSLTCLVKGFYPSDIAVEWE
SNGQPENNYKTTPPVLDSDGSFFLYSRLTVDKSRWQEGNVFSCSVLHEALH
NHYTQKSLSLSLGKIEGRMDDACPWAVSGARASPGSAASPRLREGPELSPD
DPAGLLDLRQGMFAQLVAQNVLLIDGPLSWYSDPGLAGVSLIGGLSYKEDT
KELVVAKAGVYYVFFQLELRRVVAGEGSGSVSLALHLQPLRSAAGAAALAL
TVDLPPASSEARNSAFGFQGRLLHLSAGQRLGVHLHTEARARHAWQLTQGA
TVLGLFRVTPEIPAGLPSPRSE.

In embodiments, a heterologous chimeric protein useful in the present invention comprises a variant of the extracellular domain of CSF1R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CSF1R, e.g., human CSF1R.

In embodiments, the extracellular domain of CSF1R has the following amino acid sequence:

(SEQ ID NO: 62)
IPVIEPSVPELVVKPGATVTLRCVGNGSVEWDGPPSPHWTLYSDGSSSILS
TNNATFQNTGTYRCTEPGDPLGGSAAIHLYVKDPARPWNVLAQEVVVFEDQ
DALLPCLLTDPVLEAGVSLVRVRGRPLMRHTNYSFSPWHGFTIHRAKFIQS
QDYQCSALMGGRKVMSISIRLKVQKVIPGPPALTLVPAELVRIRGEAAQIV
CSASSVDVNFDVFLQHNNTKLAIPQQSDFHNNRYQKVLTLNLDQVDFQHAG
NYSCVASNVQGKHSTSMFFRVVESAYLNLSSEQNLIQEVTVGEGLNLKVMV
EAYPGLQGFNWTYLGPFSDHQPEPKLANATTKDTYRHTFTLSLPRLKPSEA
GRYSFLARNPGGWRALTFELTLRYPPEVSVIWTFINGSGTLLCAASGYPQP
NVTWLQCSGHTDRCDEAQVLQVWDDPYPEVLSQEPFHKVTVQSLLTVETLE
HNQTYECRAHNSVGSGSWAFIPISAGAHTHPPDEFLFTP.

In embodiments, a heterologous chimeric protein comprises a variant of the extracellular domain of CSF1R. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the first domain of a heterologous chimeric protein comprises an amino acid sequence that is at least 95% identical to the amino acid sequence of SEQ ID NO: 62.

One of ordinary skill may select variants of the known amino acid sequence of CSF1R by consulting the literature, e.g., Tap, et al., “Structure-Guided Blockade of CSF1R Kinase in Tenosynovial Giant-Cell Tumor”, N. Engl. J. Med. 2015 Jul. 30; 373(5):428-37; Schubert et al., “Crystal structure of the tyrosine kinase domain of colony-stimulating factor-1 receptor (cFMS) in complex with two inhibitors.” J. Biol. Chem. 282 (6), 4094-4101 (2007); Walter et al., “The 2.7 A crystal structure of the autoinhibited human c-Fms kinase domain.” J. Mol. Biol. 367 (3), 839-847 (2007); and Mashkani et al., “Colony stimulating factor-1 receptor as a target for small molecule inhibitors”, Bioorg. Med. Chem. 18 (5), 1789-1797 (2010), each of which is incorporated by reference in its entirety.

In embodiments, a heterologous chimeric protein useful in the present invention comprises a variant of the extracellular domain of CD40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CD40L, e.g., human CD40L.

In embodiments, the extracellular domain of CD40L has the following amino acid sequence:

(SEQ ID NO: 63)
HRRLDKIEDERNLHEDFVFMKTIQRCNTGERSLSLLNCEEIKSQFEGFVKD
IMLNKEETKKENSFEMQKGDQNPQIAAHVISEASSKTTSVLQWAEKGYYTM
SNNLVTLENGKQLTVKRQGLYYIYAQVTFCSNREASSQAPFIASLCLKSPG
RFERILLRAANTHSSAKPCGQQSIHLGGVFELQPGASVFVNVTDPSQVSHG
TGFTSFGLLKL.

In embodiments, a heterologous chimeric protein comprises a variant of the extracellular domain of CD40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the second domain of a heterologous chimeric protein comprises an amino acid sequence that is at least 95% identical to the amino acid sequence of SEQ ID NO: 63.

One of ordinary skill may select variants of the known amino acid sequence of CD40L by consulting the literature, e.g. Oganesyan V., et al., “Fibronectin type III domains engineered to bind CD40L: cloning, expression, purification, crystallization and preliminary X-ray diffraction analysis of two complexes”, Acta Crystallogr Sect F Struct Biol Cryst Commun. 2013 September; 69(Pt 9):1045-8; Hollenbaugh, et al., “The human T cell antigen gp39, a member of the TNF gene family, is a ligand for the CD40 receptor expression of a soluble form of gp39 with B cell co-stimulatory activity.” EMBO J. 11 (12), 4313-4321 (1992); Gauchat et al., “Human CD40-ligand: molecular cloning, cellular distribution and regulation of expression by factors controlling IgE production.” FEBS Lett. 315 (3), 259-266 (1993); Karpusas et al., “A crystal structure of an extracellular fragment of human CD40 ligand.” Structure 3 (10), 1031-1039 (1995); and Singh et al., “The role of polar interactions in the molecular recognition of CD40L with its receptor CD40.” Protein Sci. 7 (5), 1124-1135 (1998), each of which is incorporated by reference in its entirety.

In embodiments, a heterologous chimeric protein useful in the present invention comprises: (a) a first domain comprising the amino acid sequence of SEQ ID NO: 62, (b) a second domain comprises the amino acid sequence of SEQ ID NO: 63, and (c) a linker comprises an amino acid sequence that is at least 95% identical to SEQ ID NO: 1, SEQ ID NO: 2, or SEQ ID NO: 3. Such a heterologous chimeric protein may be referred to as “CSF1R-Fc-CD40L”.

In embodiments, a CSF1R-Fc-CD40L chimeric protein used in the present invention and has the following amino acid sequence:

(SEQ ID NO: 64)
IPVIEPSVPELVVKPGATVTLRCVGNGSVEWDGPPSPHWTLYSDGSSSILS
TNNATFQNTGTYRCTEPGDPLGGSAAIHLYVKDPARPWNVLAQEVVVFEDQ
DALLPCLLTDPVLEAGVSLVRVRGRPLMRHTNYSFSPWHGFTIHRAKFIQS
QDYQCSALMGGRKVMSISIRLKVQKVIPGPPALTLVPAELVRIRGEAAQIV
CSASSVDVNFDVFLQHNNTKLAIPQQSDFHNNRYQKVLTLNLDQVDFQHAG
NYSCVASNVQGKHSTSMFFRVVESAYLNLSSEQNLIQEVTVGEGLNLKVMV
EAYPGLQGFNWTYLGPFSDHQPEPKLANATTKDTYRHTFTLSLPRLKPSEA
GRYSFLARNPGGWRALTFELTLRYPPEVSVIWTFINGSGTLLCAASGYPQP
NVTWLQCSGHTDRCDEAQVLQVWDDPYPEVLSQEPFHKVTVQSLLTVETLE
HNQTYECRAHNSVGSGSWAFIPISAGAHTHPPDEFLFTPSKYGPPCPPCPA
PEFLGGPSVFLFPPKPKDQLMISRTPEVTCVVVDVSQEDPEVQFNWYVDGV
EVHNAKTKPREEQFNSTYRVVSVLTVLHQDWLSGKEYKCKVSSKGLPSSIE
KTISNATGQPREPQVYTLPPSQEEMTKNQVSLTCLVKGFYPSDIAVEWESN
GQPENNYKTTPPVLDSDGSFFLYSRLTVDKSRWQEGNVFSCSVLHEALHNH
YTQKSLSLSLGKIEGRMDHRRLDKIEDERNLHEDFVFMKTIQRCNTGERSL
SLLNCEEIKSQFEGFVKDIMLNKEETKKENSFEMQKGDQNPQIAAHVISEA
SSKTTSVLQWAEKGYYTMSNNLVTLENGKQLTVKRQGLYYIYAQVTFCSNR
EASSQAPFIASLCLKSPGRFERILLRAANTHSSAKPCGQQSIHLGGVFELQ
PGASVFVNVTDPSQVSHGTGFTSFGLLKL.

In embodiments, the chimeric protein is capable of providing a sustained immunomodulatory effect.

In embodiments, wherein the linker is a polypeptide selected from a flexible amino acid sequence, an IgG hinge region, and an antibody sequence.

In embodiments, the linker is not a single amino acid linker, e.g., without limitation, the linker is greater than one amino acid long. In embodiments, the linker has a length of greater than 1-6 amino acids, e.g., without limitation, the linker is greater than seven amino acids long. In embodiments, the linker comprises more than a single glycine residue.

In embodiments, heterologous chimeric protein useful in the present invention comprises a linker domain adjoining the first and second domains of the protein.

In embodiments, the linker comprises at least one cysteine residue capable of forming a disulfide bond and/or comprises a hinge-CH2-CH3 Fc domain, e.g., derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In a heterologous chimeric protein useful in the present invention, the heterologous chimeric protein is a recombinant fusion protein, e.g., a single polypeptide having the extracellular domains disclosed herein. For example, in embodiments, the heterologous chimeric protein is translated as a single unit in a prokaryotic cell, a eukaryotic cell, or a cell-free expression system.

In embodiments, the present heterologous chimeric protein is producible in a mammalian host cell (i.e., an engineered T cell of the present invention) as a secretable and fully functional single polypeptide chain.

In embodiments, heterologous chimeric protein refers to a recombinant protein of multiple polypeptides, e.g., multiple extracellular domains disclosed herein, that are combined (via covalent or no-covalent bonding) to yield a single unit, e.g., in vitro (e.g., with one or more synthetic linkers disclosed herein).

Heterologous chimeric proteins useful in the present invention have a first domain which is sterically capable of binding its ligand/receptor and a second domain which is sterically capable of binding its ligand/receptor. This means that there is sufficient overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is herein referred to as “slack”) may be normally present in the extracellular domain(s), normally present in the linker, and/or normally present in the chimeric protein (as a whole). Alternately, or additionally, the chimeric protein may be modified by including one or more additional amino acid sequences (e.g., the joining linkers described below) or synthetic linkers (e.g., a polyethylene glycol (PEG) linker) which provide additional slack needed to avoid steric hindrance.

In any herein-disclosed aspect and embodiment, the heterologous chimeric protein useful in the present invention may comprise an amino acid sequence having one or more amino acid mutations relative to any of the protein sequences disclosed herein. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyic acid, α-amino isobutyric acid, 4-aminobutyric acid, Abu, 2-amino butyric acid, γ-Abu, ε-AhX, 6-amino hexanoic acid, Aib, 2-amino isobutyric acid, 3-amino propionic acid, omithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino adds, designer amino acids such as β methyl amino acids, C α-methyl amino acids, N α-methyl amino acids, and amino acid analogs in general).

Mutations may also be made to the nucleotide sequences of the heterologous chimeric proteins by reference to the genetic code, including taking into account codon degeneracy.

In embodiments, a heterologous chimeric protein useful in the present invention is capable of binding human ligand(s)/receptor(s).

In embodiments, a heterologous chimeric protein useful in the present invention is capable of binding murine ligand(s)/receptor(s).

In embodiments, each extracellular domain (or variant thereof) of the heterologous chimeric protein binds to its cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each extracellular domain (or variant thereof) of the heterologous chimeric protein binds to its cognate receptor or ligand with a K_{D} of less than about 1 μM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the heterologous chimeric protein binds to human CSF1 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

As used herein, a variant of an extracellular domain useful in the present invention is capable of binding the receptor/ligand of a native extracellular domain. For example, a variant may include one or more mutations in an extracellular domain which do not affect its binding affinity to its receptor/ligand; alternately, the one or more mutations in an extracellular domain may improve binding affinity for the receptor/ligand; or the one or more mutations in an extracellular domain may reduce binding affinity for the receptor/ligand, yet not eliminate binding altogether. In embodiments, the one or more mutations are located outside the binding pocket where the extracellular domain interacts with its receptor/ligand. In embodiments, the one or more mutations are located inside the binding pocket where the extracellular domain interacts with its receptor/ligand, as long as the mutations do not eliminate binding altogether. Based on the skilled artisan's knowledge and the knowledge in the art regarding receptor-ligand binding, she would know which mutations would permit binding and which would eliminate binding.

In embodiments, the chimeric protein exhibits enhanced stability and protein half-life.

A heterologous chimeric protein useful in the present invention may comprise more than two extracellular domains. For example, the chimeric protein may comprise three, four, five, six, seven, eight, nine, ten, or more extracellular domains. A second extracellular domain may be separated from a third extracellular domain via a linker, as disclosed herein. Alternately, a second extracellular domain may be directly linked (e.g., via a peptide bond) to a third extracellular domain. In embodiments, a heterologous chimeric protein includes extracellular domains that are directly linked and extracellular domains that are indirectly linked via a linker, as disclosed herein.

Linkers

In embodiments, the heterologous chimeric protein useful in the present invention comprises a linker.

In embodiments, the linker comprises at least one cysteine residue capable of forming a disulfide bond. The at least one cysteine residue is capable of forming a disulfide bond between a pair (or more) of heterologous chimeric proteins. Without wishing to be bound by theory, such disulfide bond forming is responsible for maintaining a useful and/or necessary multimeric state of the heterologous chimeric proteins. These allow for efficient production of the functional heterologous chimeric proteins (e.g., which do not form into non-functioning protein aggregates) and for desired activity in vitro and in vivo.

In a heterologous chimeric protein useful in the present invention, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker useful in the present invention comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a heterologous chimeric protein useful in the present invention, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NOs: 4-50 (or a variant thereof). In embodiments, the linker comprises two or more joining linkers each joining linker independently selected from SEQ ID NOs: 4-50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present heterologous chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, praline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, I253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an I253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NOs: 4 to 50, or variants thereof are located between an extracellular domain as disclosed herein and an Fc domain as disclosed herein.

In embodiments, the present heterologous chimeric proteins may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 4 to 50.

In embodiments, the first and second joining linkers may be different or they may be the same.

Without wishing to be bound by theory, including a linker comprising at least a part of an Fc domain in a heterologous chimeric protein, helps avoid formation of insoluble and, likely, non-functional protein concatamers and/or aggregates. This is in part due to the presence of cysteines in the Fc domain which are capable of forming disulfide bonds between heterologous chimeric proteins.

In embodiments, a heterologous chimeric protein may comprise one or more joining linkers, as disclosed herein, and lack an Fc domain linker, as disclosed herein.

In embodiments, the first and/or second joining linkers are independently selected from the amino acid sequences of SEQ ID NOs: 4 to 50 and are provided in Table 1 below:

TABLE 1
Illustrative linkers (Fc domain linkers and joining linkers)

| SEQ | |
|---|---|
| ID | |
| NO. | Sequence |
| 1 | APEFLGGPSVFLFPPKPKDTLMISRTPEVTCVVVDVSQEDPEVQFNWYVDGVEVHNAKTKPREEQFNS |
| | TYRVVSVLTVLHQDWLSGKEYKCKVSSKGLPSSIEKTISNATGQPREPQVYTLPPSQEEMTKNQVSLTC |
| | LVKGFYPSDIAVEWESNGQPENNYKTTPPVLDSDGSFFLYSRLTVDKSSWQEGNVFSCSVMHEALHN |
| | HYTQKSLSLSLGK |
| 2 | APEFLGGPSVFLFPPKPKDQLMISRTPEVTCVVVDVSQEDPEVQFNWYVDGVEVHNAKTKPREEQFNS |
| | TYRVVSVLTTPHSDWLSGKEYKCKVSSKGLPSSIEKTISNATGQPREPQVYTLPPSQEEMTKNQVSLTC |
| | LVKGFYPSDIAVEWESNGQPENNYKTTPPVLDSDGSFFLYSRLTVDKSSWQEGNVFSCSVLHEALHNH |
| | YTQKSLSLSLGK |
| 3 | APEFLGGPSVFLFPPKPKDQLMISRTPEVTCVVVDVSQEDPEVQFNWYVDGVEVHNAKTKPREEQFNS |
| | TYRVVSVLTVLHQDWLSGKEYKCKVSSKGLPSSIEKTISNATGQPREPQVYTLPPSQEEMTKNQVSLTC |
| | LVKGFYPSDIAVEWESNGQPENNYKTTPPVLDSDGSFFLYSRLTVDKSRWQEGNVFSCSVLHEALHNH |
| | YTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGSGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42). A(EAAAK)₄ALEA(EAAAK)₄A (SEQ ID NO: 43). PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS. In embodiments, a joining linker has the sequence (Gly)ₙ, where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47 GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

The combination of a first joining linker, an Fc Domain linker, and a second joining linker is referend to herein as a “modular linker”. In embodiments, a heterologous chimeric protein comprises a modular linker as shown in Table 2:

TABLE 2
Illustrative modular linkers

| | | Joining | Modular Linker = Joining Linker |
|---|---|---|---|
| Joining Linker 1 | Fc | Linker 2 | 1 + Fc + Joining Linker 2 |
| SKYGPPCPSCP | APEFLGGPSVFLFPPKPKDTLMIS | IEGRMD | SKYGPPCPSCPAPEFLGGPSVFL |
| (SEQ ID NO: 4) | RTPEVTCVVVDVSQEDPEVQFN | (SEQ ID NO: 7) | FPPKPKDTLMISRTPEVTCVVVDV |
| | WYVDGVEVHNAKTKPREEQFNS | | SQEDPEVQFNWYVDGVEVHNAK |
| | TYRVVSVLTVLHQDWLSGKEYKC | | TKPREEQFNSTYRVVSVLTVLHQ |
| | KVSSKGLPSSIEKTISNATGQPRE | | DWLSGKEYKCKVSSKGLPSSIEK |
| | PQVYTLPPSQEEMTKNQVSLTCL | | TISNATGQPREPQVYTLPPSQEE |
| | VKGFYPSDIAVEWESNGQPENNY | | MTKNQVSLTCLVKGFYPSDIAVE |
| | KTTPPVLDSDGSFFLYSRLTVDKS | | WESNGQPENNYKTTPPVLDSDG |
| | SWQEGNVFSCSVMHEALHNHYT | | SFFLYSRLTVDKSSWQEGNVFSC |
| | QKSLSLSLGK (SEQ ID NO: 1) | | SVMHEALHNHYTQKSLSLSLGKIE |
| | | | GRMD (SEQ ID NO: 51) |
| SKYGPPCPSCP | APEFLGGPSVFLFPPKPKDQLMIS | IEGRMD | SKYGPPCPSCPAPEFLGGPSVFL |
| (SEQ ID NO: 4) | RTPEVTCVVVDVSQEDPEVQFN | (SEQ ID NO: 7) | FPPKPKDQLMISRTPEVTICVVVD |
| | WYVDGVEVHNAKTKPREEQFNS | | VSQEDPEVQFNWYVDGVEVHNA |
| | TYRVVSVLTTPHSDWLSGKEYKC | | KTKPREEQFNSTYRVVSVLTTPH |
| | KVSSKGLPSSIEKTISNATGQPRE | | SDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | SWQEGNVFSCSVLHEALHNHYT | | GSFFLYSRLTVDKSSWQEGNVFS |
| | QKSLSLSLGK (SEQ ID NO: 2) | | CSVLHEALHNHYTQKSLSLSLGKI |
| | | | EGRMD (SEQ ID NO: 52) |
| SKYGPPCPSCP | APEFLGGPSVFLFPPKPKDQLMIS | IEGRMD | SKYGPPCPSCPAPEFLGGPSVFL |
| (SEQ ID NO: 4) | RTPEVTCVVVDVSQEDPEVQFN | (SEQ ID NO: 7) | FPPKPKDQLMISRTPEVTCVVVD |
| | WYVDGVEVHNAKTKPREEQFNS | | VSQEDPEVQFNWYVDGVEVHNA |
| | TYRVVSVLTVLHQDWLSGKEYKC | | KTKPREEQFNSTYRVVSVLTVLH |
| | KVSSKGLPSSIEKTISNATGQPRE | | QDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | RWQEGNVFSCSVLHEALHNHYT | | GSFFLYSRLTVDKSRWQEGNVFS |
| | QKSLSLSLGK (SEQ ID NO: 3) | | CSVLHEALHNHYTQKSLSLSLGKI |
| | | | EGRMD (SEQ ID NO: 53) |
| SKYGPPCPPCP | APEFLGGPSVFLFPPKPKDTLMIS | IEGRMD | SKYGPPCPPCPAPEFLGGPSVFL |
| (SEQ ID NO: 5) | RTPEVTCVVVDVSQEDPEVQFN | (SEQ ID NO: 7) | FPPKPKDTLMISRTPEVTCVVVDV |
| | WYVDGVEVHNAKTKPREEQFNS | | SQEDPEVQFNWYVDGVEVHNAK |
| | TYRVVSVLTVLHQDWLSGKEYKC | | TKPREEQFNSTYRVVSVLTVLHQ |
| | KVSSKGLPSSIEKTISNATGQPRE | | DWLSGKEYKCKVSSKGLPSSIEK |
| | PQVYTLPPSQEEMTKNQVSLTCL | | TISNATGQPREPQVYTLPPSQEE |
| | VKGFYPSDIAVEWESNGQPENNY | | MTKNQVSLTCLVKGFYPSDIAVE |
| | KTTPPVLDSDGSFFLYSRLTVDKS | | WESNGQPENNYKTTPPVLDSDG |
| | SWQEGNVFSCSVMHEALHNHYT | | SFFLYSRLTVDKSSWQEGNVFSC |
| | QKSLSLSLGK (SEQ ID NO: 1) | | SVMHEALHNHYTQKSLSLSLGKIE |
| | | | GRMD (SEQ ID NO: 54) |
| SKYGPPCPPCP | APEFLGGPSVFLFPPKPKDQLMIS | IEGRMD | SKYGPPCPPCPAPEFLGGPSVFL |
| (SEQ ID NO: 5) | RTPEVTCVVVDVSQEDPEVQFN | (SEQ ID NO: 7) | FPPKPKDQLMISRTPEVTCVVVD |
| | WYVDGVEVHNAKTKPREEQFNS | | VSQEDPEVQFNWYVDGVEVHNA |
| | TYRVVSVLTTPHSDWLSGKEYKC | | KTKPREEQFNSTYRWSVLTTPH |
| | KVSSKGLPSSIEKTISNATGQPRE | | SDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | SWQEGNVFSCSVLHEALHNHYT | | GSFFLYSRLTVDKSSWQEGNVFS |
| | QKSLSLSLGK (SEQ ID NO: 2) | | CSVLHEALHNHYTQKSLSLSLGKI |
| | | | EGRMD (SEQ ID NO: 55) |
| SKYGPPCPPCP | APEFLGGPSVFLFPPKPKDQLMIS | IEGRMD | SKYGPPCPPCPAPEFLGGPSVFL |
| (SEQ ID NO: 5) | RTPEVTCVVVDVSQEDPEVQFN | (SEQ ID NO: 7) | FPPKPKDQLMISRTPEVTCVVVD |
| | WYVDGVEVHNAKTKPREEQFNS | | VSQEDPEVQFNWYVDGVEVHNA |
| | TYRVVSVLTVLHQDWLSGKEYKC | | KTKPREEQFNSTYRVVSVLTVLH |
| | KVSSKGLPSSIEKTISNATGQPRE | | QDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | RWQEGNVFSCSVLHEALHNHYT | | GSFFLYSRLTVDKSRWQEGNVFS |
| | QKSLSLSLGK (SEQ ID NO: 3) | | CSVLHEALHNHYTQKSLSLSLGKI |
| | | | EGRMD (SEQ ID NO: 56) |

In embodiments, the present heterologous chimeric proteins may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90% or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 51 to 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + soluble |
| GGSG (SEQ ID NO: 13) | Flexible |
| GGSGGGSGGGSG (SEQ ID NO: 14) | Flexible |
| EAAAKEAAAKEAAAK (SEQ ID NO: 15) | Rigid Alpha Helix |
| EAAAREAAAREAAAREAAAR | Rigid Alpha Helix |
| (SEQ ID NO: 16) | |
| GGGGSGGGGSGGGGSAS | Flexible |
| (SEQ ID NO: 17) | |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP | Rigid |
| (SEQ ID NO: 23) | |

In embodiments, the linker may be functional. For example, without limitation, the linker may function to improve the folding and/or stability, improve the expression, improve the pharmacokinetics, and/or improve the bioactivity of the present heterologous chimeric protein. In another example, the linker may function to target the heterologous chimeric protein to a particular cell type or location.

In embodiments, a heterologous chimeric protein comprises only one joining linkers.

In embodiments, a heterologous chimeric protein lacks joining linkers.

In embodiments, the linker is a synthetic linker such as polyethylene glycol (PEG).

In embodiments, a heterologous chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

Engineered T Cells

Disclosed herein is an engineered T cell that expresses a chimeric antigen receptor and a heterologous chimeric protein. The chimeric antigen receptor comprises an antigen-binding domain, a transmembrane domain, and an intracellular domain, which comprises a costimulatory domain and/or a signaling domain. The heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein. FIG. 1 illustrates an engineered T cell of the present invention.

In embodiments, the first domain of the heterologous chimeric protein comprises substantially the entire extracellular domain of the first transmembrane protein and/or the second domain of the heterologous chimeric protein comprises substantially the entire extracellular domain of the second transmembrane protein.

In embodiments, the first domain is capable of binding a ligand/receptor of the first transmembrane protein and the second domain is capable of binding a ligand/receptor of the second transmembrane protein.

In embodiments, the first domain is capable of inhibiting an immunosuppressive signal when bound to its ligand/receptor and/or the second domain is capable of activating an immune stimulatory signal when bound to its ligand/receptor.

In embodiments, the ligand/receptor of the first transmembrane protein is expressed by a cancer cell and/or the ligand/receptor of the second transmembrane protein is expressed by the engineered T cell and/or by a native T cell.

In embodiments, the heterologous chimeric protein is secreted by the engineered T cell. In embodiments, the heterologous chimeric protein is secreted in response to activation of the chimeric antigen receptor expressed by the engineered T cell. In an embodiment, activation of the chimeric antigen receptor stimulates a chimeric antigen receptor-associated promoter such as a nuclear factor of activated T cells (NFAT) promoter.

In embodiments, the chimeric antigen receptor comprises an antigen-binding domain, a transmembrane domain, and an intracellular domain, which comprises a costimulatory domain and/or a signaling domain.

In embodiments, the antigen-binding domain of the chimeric antigen receptor is capable of binding an antigen expressed by a cancer cell, e.g., a human cancer cell-specific antigen.

In embodiments, the intracellular domain of a chimeric antigen receptor comprises one or both of a costimulatory domain and a signaling domain.

In embodiments, the heterologous chimeric protein and/or chimeric antigen receptor is capable of increasing or preventing a decrease in a sub-population of CD4+ and/or CD8+ T cells.

In embodiments, the heterologous chimeric protein increases the number of non-engineered T cell clones present near the cancer cells/the tumor site.

The heterologous chimeric protein is capable of forming a stable synapse between a cancer cell and the engineered T cell when its first domain is bound to the ligand/receptor of the first transmembrane protein expressed by the cancer cell and the second domain is bound to the ligand/receptor of the second transmembrane protein expressed by the engineered T cell; and/or the chimeric antigen receptor is capable of forming a stable synapse between the cancer cell and the engineered T cell when bound to the antigen expressed by the cancer cell. The stable synapse provides a spatial orientation that favors an anti-cancer effect/tumor reduction by the engineered T cell. In embodiments, the spatial orientation positions the engineered T cell to attack the cancer cell. In embodiments, the stable synapse allows for proliferation of the engineered T cell near the cancer cell. In embodiments, the stable synapse allows for recruitment and proliferation of non-engineered, native, T cells near the cancer cells/the tumor site. In embodiments, the stable synapse allows sufficient signal transmission to provide expression and/or release of a stimulatory signal (e.g., a cytokine).

In embodiments, the heterologous chimeric protein expressed and secreted by an engineered T cell of the present invention is selected from Table 4 below.

TABLE 4
Illustrative Heterologous Chimeric Proteins

BTLA-Fc-4-1BBL
BTLA-Fc-GITRL
BTLA-Fc-LIGHT
BTLA-Fc-OX40L
BTLA-Fc-TL1A
CD172a-Fc-CD40L
CD172a-Fc-LIGHT
CSF1R-Fc-CD40L
CTLA4-Fc-TL1A
LAG3-Fc-4-1BBL
LAG3-Fc-CD40L
LAG3-Fc-GITRL
LAG3-Fc-LIGHT
LAG3-Fc-OX40L
PD-1-Fc-4-1BBL
PD-1-Fc-CD40L
PD-1-Fc-GITRL
PD-1-Fc-LIGHT
PD-1-Fc-OX40L
PD-1-Fc-TL1A
PD-1-Fc-TRAIL
TIGIT-Fc-4-1BBL
TIGIT-Fc-GITRL
TIGIT-Fc-LIGHT
TIGIT-Fc-OX40L
TIGIT-Fc-TL1A
TIM3-Fc-4-1BBL
TIM3-Fc-CD40L
TIM3-Fc-GITRL
TIM3-Fc-LIGHT
TIM3-Fc-OX40L
TIM3-Fc-TL1A
TMIGD2-Fc-OX40L
VSIG8-Fc-CD40L
VSIG8-Fc-GITRL
VSIG8-Fc-LIGHT
VSIG8-Fc-OX40L
VSIG8-Fc-TL1A

In embodiments, the chimeric antigen receptor expressed by an engineered T cell of the present invention is selected from Table 5 below:

TABLE 5
Illustrative Chimeric Antigen Receptor (CAR) T cell Targets

BCMA
CD10
CD19
CD20
CD22
CD30
CD33
CD34
CD38
CD56
CD80/86
CD117
CD123
CD123
CD133
CD138
CEA
CS1/CD319/SLAM
F7
disialoganglioside
(GD2)
EphA2
EGFR
EpCAM
GPC3
HER2
integrin β7
Lewis-Y
Mesothelin
MUC1
PD-L1
PSCA

In embodiments, a herein-disclosed engineered T cell expresses and secretes one or more heterologous chimeric proteins from Table 4 and expresses one or more chimeric antigen receptors from Table 5. In embodiments, an engineered T cell expresses and secretes one heterologous chimeric protein from Table 4 and expresses more than one chimeric antigen receptor from Table 5. In embodiments, an engineered T cell expresses and secretes more than one heterologous chimeric protein from Table 4 and expresses one chimeric antigen receptor from Table 5. In embodiments, an engineered T cell expresses and secretes more than one heterologous chimeric protein from Table 4 and expresses more than one chimeric antigen receptor from Table 5.

As examples, an engineered T cell expresses and secretes the PD-1-Fc-OX40L heterologous chimeric protein and/or the PD-1-Fc-OX40L heterologous chimeric protein and expresses a chimeric antigen receptor that binds CD19 that is presented by a cancer cell and/or an engineered T cell expresses and secretes the CSF1R-Fc-CD40L heterologous chimeric protein and expresses a chimeric antigen receptor that binds CD19 presented by a cancer cell.

The engineered T cells of the herein-disclosed subject matter can be cells of the lymphoid lineage. The lymphoid lineage, comprising B and T cells, provides for the production of antibodies, regulation of the cellular immune system, detection of foreign agents in the blood, detection of cells foreign to the host, and the like. Non-limiting examples of engineered T cells of the lymphoid lineage include T cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the herein-disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Mucosal associated invariant T cells, and γδ T cells. Cytotoxic T cells (CTL or killer T cells) are a subset of T lymphocytes capable of inducing the death of infected somatic or cancer cells. A patients own T cells may be genetically modified to target specific antigens through the introduction of a CAR.

Types of human lymphocytes of the present invention include, without limitation, peripheral donor lymphocytes genetically modified to express CARs (Sadelain, M., et al. 2003 Nat Rev Cancer 3:35-45), peripheral donor lymphocytes genetically modified to express a full-length antigen-recognizing T cell receptor complex comprising the α and β heterodimer (Morgan, R. A., et al. 2006 Science 314: 126-129), lymphocyte cultures derived from tumor infiltrating lymphocytes (TILs) in tumor biopsies (Panelli, M. C., et al. 2000 J Immunol 164:495-504; Panelli, M. C., et al. 2000 J Immunol 164:4382-4392), and selectively in vitro-expanded antigen-specific peripheral blood leukocytes employing artificial antigen-presenting cells (aAPCs) or pulsed dendritic cells (Dupont, J., et al. 2005 Cancer Res 65:5417-5427; Papanicolaou, G. A., et al. 2003 Blood 102:2498-2505). The T cells may be autologous, allogeneic, or derived in vitro from engineered progenitor or stem cells.

In embodiments, the engineered T cell is a CD4+ T cell or a CD8+ T cell.

In embodiments, the engineered T cell expresses an alpha/beta T cell receptor.

In embodiments, the engineered T cell expresses a gamma/delta T cell receptor.

In embodiments, the engineered T cell is derived from an allogeneic T cell, an autologous T cell, or a tumor-infiltrating lymphocyte (TIL). In embodiments, the engineered T cell is a human cell.

In embodiments, the engineered T cell is derived from a T cell or T-cell progenitor that was obtained from the subject, e.g., a subject in need of an anti-cancer therapy. In embodiments, the T cell or T-cell progenitor is obtained from an autologous source (e.g., from the same subject as in need of an anti-cancer therapy). In embodiments, the autologous T cell or T-cell progenitor is obtained from a subject and cultured and expanded in vitro. In embodiments, the cultured and/or expanded the autologous T cell or T-cell progenitor are engineered to express the chimeric T cell receptor and/or the heterologous chimeric protein as disclosed herein. In embodiments, the autologous T cell or T-cell progenitor are engineered to express the chimeric T cell receptor and/or the heterologous chimeric protein are analyzed using methods disclosed herein. In embodiments, the autologous T cell or T-cell progenitor are engineered to express the chimeric T cell receptor and/or the heterologous chimeric protein are optionally cultured in vitro and/or expanded, and administered to a subject in need thereof. In embodiments, the autologous T cell or T-cell progenitor are engineered to express the chimeric T cell receptor and/or the heterologous chimeric protein are manufactured as disclosed herein, optionally analyzed using methods disclosed herein, and administered to a subject in need thereof.

In embodiments, the T cell or T-cell progenitor is obtained from an allogeneic source of isolated human T cells, namely TCR-deficient T cells, that can be manufactured in advance of patient need and inexpensively. In embodiments, the T cell or T-cell progenitor is obtained from an allogeneic source allow the creation of a single therapeutic product at a single site. In embodiments, the allogeneic T cells that are modified as disclosed herein do not express functional T cell receptors (TCRs). Without being bound by theory, it is to be understood that some, or even all, of the TCR subunits/dimers may be expressed on the cell surface, but that the T cell does not express enough functional TCR to induce an undesirable reaction in the host. Without functional TCRs on their surface, the allogeneic T cells fail to mount an undesired immune response to host cells. In embodiments, the TCR-deficient allogeneic T cells that are modified as disclosed herein, fail to cause graft versus host disease (GVHD), for example, as they cannot recognize the host MHC molecules. Additionally, these TCR-deficient T cells can be engineered to simultaneously express functional, non-TCR, disease-specific receptors.

As is well known to one of skill in the art, various methods are readily available for isolating allogeneic T cells from a subject. For example, using cell surface marker expression or using commercially available kits (e.g., ISOCELL from Pierce, Rockford, Ill.).

In embodiments, the allogeneic T cell or T-cell progenitor is obtained from a subject and cultured and expanded in vitro. In embodiments, the cultured and/or expanded the allogeneic T cell or T-cell progenitor are engineered to express the chimeric T cell receptor and/or the heterologous chimeric protein as disclosed herein. In embodiments, the allogeneic T cell or T-cell progenitor are engineered to express the chimeric T cell receptor and/or the heterologous chimeric protein are analyzed using methods disclosed herein. In embodiments, the allogeneic T cell or T-cell progenitor are engineered to express the chimeric T cell receptor and/or the heterologous chimeric protein are optionally cultured in vitro and/or expanded, and administered to a subject in need thereof. In embodiments, the allogeneic T cell or T-cell progenitor are engineered to express the chimeric T cell receptor and/or the heterologous chimeric protein are manufactured as disclosed herein, optionally analyzed using methods disclosed herein, and administered to a subject in need thereof.

Method of Manufacturing Engineered T Cells

An aspect of the present invention is a method for manufacturing an engineered T cell. The method comprises steps of: (i) obtaining a T-cell or T-cell progenitor cell from a subject; (ii) transfecting the T-cell or T-cell progenitor cell with a first DNA molecule encoding a chimeric antigen receptor and designed to be integrated into the genome of the T cell or T-cell progenitor; and (iii) transfecting the T-cell or T-cell progenitor cell with a second DNA molecule encoding a heterologous chimeric protein and designed to be integrated into the genome of the T cell or T-cell progenitor. The heterologous chimeric protein of this aspect comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein. In embodiments, step (ii) precedes step (iii). In embodiments, step (iii) precedes step (ii). In embodiments, step (ii) and step (iii) are contemporaneous.

Another aspect of the present invention is another method for manufacturing an engineered T cell. The method comprising steps of: (i) obtaining a T-cell or T-cell progenitor cell that has been transfected with a first DNA molecule encoding a chimeric antigen receptor and designed to be integrated into the genome of the T cell or T-cell progenitor; and (ii) transfecting the T cell or T-cell progenitor cell of step (i) with a second DNA molecule encoding a heterologous chimeric protein and designed to be integrated into the genome of the T cell or T-cell progenitor. In this aspect, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

Yet another aspect of the present invention is yet another method for manufacturing an engineered T cell. The method comprises steps of: (i) obtaining a T-cell or T-cell progenitor cell that has been transfected with a first DNA molecule encoding a heterologous chimeric protein and designed to be integrated into the genome of the T cell or T-cell progenitor and (ii) transfecting the T cell or T-cell progenitor cell of step (i) with a second DNA molecule encoding a chimeric antigen receptor and designed to be integrated into the genome of the T cell or T-cell progenitor. In this aspect, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein.

In yet another aspect, the present invention provides yet another method for manufacturing an engineered T cell. The method comprises steps of: (i) obtaining a T-cell or T-cell progenitor cell, and (ii) transfecting the T cell or T-cell progenitor cell of step (i) with a DNA molecule encoding a chimeric antigen receptor and designed to be integrated into the genome of the T cell or T-cell progenitor. In this aspect, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein. In embodiments, the T cell is a T-cell progenitor cell. In embodiments, the T-cell or T-cell progenitor cell is obtained from a patient. In embodiments, the T-cell or T-cell progenitor cell is specific to a cancer antigen.

These aspects comprise the manufacture of an engineered T cell that expresses any of the herein-disclosed chimeric antigen receptors and any of the herein-disclosed heterologous chimeric proteins.

In embodiments, the chimeric antigen receptor is encoded by a nucleic acid introduced into a genomic safe harbor (GSR) in the engineered T cell's genome. In embodiments, the GSR is the adeno-associated virus site 1 (AAVS1); the chemokine (C-C motif) receptor 5 (CCR5) gene; or the Rosa26 locus/human orthologue of the Rosa26 locus.

In embodiments, the chimeric antigen receptor is encoded by a nucleic acid introduced into an endogenous T cell receptor locus in the T cell's genome.

Any targeted genome editing methods can be used to integrate a nucleic acid encoding a chimeric antigen receptor into a selected locus of the genome of an engineered T cell. In embodiments, the expression of the chimeric antigen receptor is driven by an endogenous promoter/enhancer within or near the locus. In embodiments, the expression of the chimeric antigen receptor is driven by an exogenous promoter integrated into the locus. The locus where the chimeric antigen receptor is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In embodiments, the clustered regularly interspaced short palindromic repeats (CRISPR) system is used to integrate a nucleic acid encoding a chimeric antigen receptor in selected loci of the genome of an engineered T cell. Methods using the CRISPR system are described, for example, in WO2014093661, WO2015123339, and WO2015089354, each of which is incorporated by reference in its entirety.

In embodiments, zinc-finger nucleases (ZFN) are used to integrate a nucleic acid encoding a chimeric antigen receptor in selected loci of the genome of an engineered T cell. Methods using the ZFN system are described, for example, in WO2009146179, WO2008060510, and CN102174576, each of which is incorporated by reference in its entirety.

In embodiments, the Transcription activator-like effector nucleases (TALEN) system is used to integrate a nucleic acid encoding a chimeric antigen receptor in selected loci of the genome of an engineered T cell. Methods using the TALEN system are described, for example, in WO2014134412, WO2013163628, and WO2014040370, each of which is incorporated by reference in its entirety.

Methods for delivering the genome-editing agents can vary depending on the need. In embodiments, the components of a selected genome editing method are delivered as DNA constructs in one or more plasmids. In embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within the selected locus, or anywhere that can influence gene expression of the integrated chimeric antigen receptor. In embodiments, the modification is introduced upstream of the transcriptional start site of the integrated chimeric antigen receptor. In embodiments, the modification is introduced between the transcriptional start site and the protein-coding region of the chimeric antigen receptor. In embodiments, the modification is introduced downstream of the protein coding region of the chimeric antigen receptor.

Expression vectors which comprise a nucleic acid encoding a herein-disclosed heterologous chimeric protein may be used or nucleic acids encoding a herein-disclosed heterologous chimeric protein may stably integrated in the genome of an engineered T cell.

In embodiments, nucleic acids encoding the three fragments (the extracellular domain of a first transmembrane protein, followed by a linker sequence, followed by the extracellular domain of a second transmembrane protein) into a vector (plasmid, viral or other).

In embodiments, nucleic acid encoding the heterologous chimeric proteins, or a complement thereof, operably linked to an expression control region, or complement thereof, that is functional in a mammalian cell.

In embodiments, the present invention contemplates the use of inducible promoters capable of effecting high level of expression transiently in response to a cue. For example, when in the proximity of a cancer cell, a cell transformed with an expression vector for the chimeric protein and/or a cell having a stable integration of the nucleic acid encoding the chimeric protein and comprising an inducible promoter is induced to transiently produce a high level of the agent by exposing the transfected cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Stable integration of a nucleic acid (encoding the chimeric antigen receptor and/or encoding the heterologous chimeric protein) may be promoted/selected for using antibiotic selection. Commonly used antibiotics for selection of stable integration include Blasticidin, G-418 (also known as Geneticin), Hygromycin, Kanamycin, Neomycin, Puromycin, and Zeocin.

There are varieties of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a cancer cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

In embodiments, nucleic acids encoding the chimeric antigen receptor and/or nucleic acids encoding the heterologous chimeric proteins can be packaged into viral vectors. Many viral vectors useful for gene therapy are known (see, e.g., Lundstrom, Trends Biotechnol., 21: 117-122, 2003. In embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244: 1275-1281, 1989; Eglitis et al., BioTechniques 6:608-614, 1988; Tolstoshev et al., Current Opinion in Biotechnology 1:55-61, 1990; Sharp, The Lancet 337: 1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346, which is hereby incorporated by reference in its entirety).

The nucleic acid encoding the chimeric antigen receptor and/or the nucleic acid encoding the heterologous chimeric proteins can be constructed in a single vectors, or in a single, multicistronic expression cassette, in multiple expression cassettes of a single vector, or in multiple vectors. Examples of elements which create polycistronic expression cassette include, but is not limited to, various Internal Ribosome Entry Sites (IRES, e.g., poliovirus IRES and encephalomyocarditis virus IRES) and 2A peptides (e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Nucleic acids encoding the chimeric antigen receptor and/or nucleic acids encoding the heterologous chimeric proteins may be transfected into a T cell or T cell progenitor ex vivo (e.g., an autologous or heterologous primary cell or progeny thereof); after which the now engineered T cell (or its descendants) is injected into a targeted tissue or is injected/infused into a patient systemically.

In embodiments, the methods of manufacturing further comprise a step of culturing the transfected T cell or T-cell progenitor cell (now referred to as an engineered T cell) in a medium that selectively enhances proliferation of chimeric antigen receptor-expressing cells and/or heterologous chimeric protein-expressing cells.

In embodiments, the methods further comprise a step of isolating an engineered T cell that expresses the chimeric antigen receptor and/or expresses the heterologous chimeric protein.

The present invention also relates to an engineered T cell manufactured by a herein-disclosed method and/or isolated by a herein-disclosed method.

Another aspect of the present invention is a population of engineered T cells obtained by a herein-disclosed method.

Populations of Engineered T Cells

Aspects of the present invention provide populations of cells comprising herein-disclosed engineered T cells and methods for obtaining the same.

Methods comprise obtaining an engineered T cell manufactured by a herein-disclosed method and culturing the engineered T cell in a medium that enhances proliferation of the engineered T cell and thereby obtaining a population of engineered T cells.

In embodiments, the population comprises an engineered T cell that expresses a chimeric antigen receptor and expresses a heterologous chimeric protein and further comprises an engineered T cell that expresses a chimeric antigen receptor and does not express a heterologous chimeric protein.

In embodiments, the population comprises an engineered T cell that expresses a chimeric antigen receptor and expresses a heterologous chimeric protein and further comprises an engineered T cell that expresses a heterologous chimeric protein and does not expresses a chimeric antigen receptor.

In embodiments, the population comprises an engineered T cell that expresses a chimeric antigen receptor and expresses a heterologous chimeric protein and further comprises an engineered T cell that expresses a heterologous chimeric protein and does not expresses a chimeric antigen receptor.

In embodiments, the population comprises an engineered T cell that expresses a chimeric antigen receptor and expresses a heterologous chimeric protein and further comprises a T cell that does not express a heterologous chimeric protein nor a chimeric antigen receptor (e.g., a native T cell or T cell progenitor).

Accordingly, a population may comprise engineered T cells that express both chimeric proteins and additional engineered T cells that express one chimeric protein and/or T cells that express neither chimeric protein.

Analysis of Engineered T Cells

In yet another aspect, the present invention provides a method of analyzing an engineered T cell. The method comprises steps of: (A) manufacturing the engineered T cell, (B) culturing the engineered T cell, (C) isolating culture supernatant of the engineered T cell, optionally enriching or partially purifying the culture supernatant, (D) contacting the culture supernatant of the engineered T cell with a second cell, and (E) analyzing expression of a cytokine or chemokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A, ACVR2B, AXL, B7-H3, BCMA, BTLA, BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA (without limitation, e.g. using reverse transcriptase polymerase chain reaction). In embodiments, the expression is analyzed by detection and/or quantitation of protein.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell from a subject; (ii) transfecting the T-cell or T-cell progenitor cell with a first DNA molecule encoding a chimeric antigen receptor and designed to be integrated into the genome of the T cell or T-cell progenitor; and (iii) transfecting the T-cell or T-cell progenitor cell with a second DNA molecule encoding a heterologous chimeric protein and designed to be integrated into the genome of the T cell or T-cell progenitor. The heterologous chimeric protein of this aspect comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein, (B) culturing the engineered T cell, (C) isolating culture supernatant of the engineered T cell, optionally enriching or partially purifying the culture supernatant, (D) contacting the culture supernatant of the engineered T cell with a second cell, and (E) analyzing expression of a cytokine or chemokine by the second cell. In embodiments, the second cell expresses a receptor for a transmembrane protein selected from 2B4, 4-1BB, ACVR1b, ACVR2A, ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A NKp46(NCR1), NKp80(KLRF1), NTB-A, OX40, PD-1, PD-1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein. In embodiments, step (ii) precedes step (iii). In embodiments, step (iii) precedes step (ii). In embodiments, step (ii) and step (iii) are contemporaneous.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell that has been transfected with a first DNA molecule encoding a chimeric antigen receptor and designed to be integrated into the genome of the T cell or T-cell progenitor; and (ii) transfecting the T cell or T-cell progenitor cell of step (i) with a second DNA molecule encoding a heterologous chimeric protein and designed to be integrated into the genome of the T cell or T-cell progenitor, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein, (B) culturing the engineered T cell, (C) isolating culture supernatant of the engineered T cell, optionally enriching or partially purifying the culture supernatant, (D) contacting the culture supernatant of the engineered T cell with a second cell, and (E) analyzing expression of a cytokine or chemokine by the second cell. In embodiments, the second cell expresses a receptor for a transmembrane protein selected from 2B4, 4-1BB, ACVR1b, ACVR2A ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell that has been transfected with a first DNA molecule encoding a heterologous chimeric protein and designed to be integrated into the genome of the T cell or T-cell progenitor and (ii) transfecting the T cell or T-cell progenitor cell of step (i) with a second DNA molecule encoding a chimeric antigen receptor and designed to be integrated into the genome of the T cell or T-cell progenitor, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein, (B) culturing the engineered T cell, (C) isolating culture supernatant of the engineered T cell, optionally enriching or partially purifying the culture supernatant, (D) contacting the culture supernatant of the engineered T cell with a second cell, and (E) analyzing expression of a cytokine or chemokine by the second cell. In embodiments, the second cell expresses a receptor for a transmembrane protein selected from 2B4, 4-1BB, ACVR1b, ACVR2A ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell, and (ii) transfecting the T cell or T-cell progenitor cell of step (i) with a DNA molecule encoding a chimeric antigen receptor and designed to be integrated into the genome of the T cell or T-cell progenitor, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein. In embodiments, the T cell is a T-cell progenitor cell. In embodiments, the T-cell or T-cell progenitor cell is obtained from a patient. In embodiments, the T-cell or T-cell progenitor cell is specific to a cancer antigen, (B) culturing the engineered T cell, (C) isolating culture supernatant of the engineered T cell, optionally enriching or partially purifying the culture supernatant, (D) contacting the culture supernatant of the engineered T cell with a second cell, and (E) analyzing expression of a cytokine or chemokine by the second cell. In embodiments, the second cell expresses a receptor for a transmembrane protein selected from 2B4, 4-1BB, ACVR1b, ACVR2A ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-71F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In yet another aspect, the present invention provides a method of analyzing an engineered T cell. The method comprises steps of: (A) manufacturing the engineered T cell, (B) co-culturing the engineered T cell with a second cell, and (C) analyzing expression of a cytokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell from a subject; (ii) transfecting the T-cell or T-cell progenitor cell with a first DNA molecule encoding a chimeric antigen receptor and designed to be integrated into the genome of the T cell or T-cell progenitor; and (iii) transfecting the T-cell or T-cell progenitor cell with a second DNA molecule encoding a heterologous chimeric protein and designed to be integrated into the genome of the T cell or T-cell progenitor. The heterologous chimeric protein of this aspect comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein, (B) co-culturing the engineered T cell with a second cell, and (C) analyzing expression of a cytokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A, ACVR2B, AXL, B7-H3, BCMA, BTLA, BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein. In embodiments, step (ii) precedes step (iii). In embodiments, step (iii) precedes step (ii). In embodiments, step (ii) and step (iii) are contemporaneous.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell that has been transfected with a first DNA molecule encoding a chimeric antigen receptor and designed to be integrated into the genome of the T cell or T-cell progenitor; and (ii) transfecting the T cell or T-cell progenitor cell of step (i) with a second DNA molecule encoding a heterologous chimeric protein and designed to be integrated into the genome of the T cell or T-cell progenitor, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein, (B) co-culturing the engineered T cell with a second cell, and (C) analyzing expression of a cytokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell that has been transfected with a first DNA molecule encoding a heterologous chimeric protein and designed to be integrated into the genome of the T cell or T-cell progenitor and (ii) transfecting the T cell or T-cell progenitor cell of step (i) with a second DNA molecule encoding a chimeric antigen receptor and designed to be integrated into the genome of the T cell or T-cell progenitor, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein, (B) co-culturing the engineered T cell with a second cell, and (C) analyzing expression of a cytokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In yet another aspect, the present invention provides a method of analyzing an engineered T cell. The method comprises steps of: (A) manufacturing the engineered T cell, wherein the manufacturing comprises steps of: (i) obtaining a T-cell or T-cell progenitor cell, and (ii) transfecting the T cell or T-cell progenitor cell of step (i) with a DNA molecule encoding a chimeric antigen receptor and designed to be integrated into the genome of the T cell or T-cell progenitor, the heterologous chimeric protein comprises the general structure: N terminus-(a)-(b)-(c)-C terminus, in which (a) is a first domain comprising an extracellular domain of a first transmembrane protein, (b) is a linker domain adjoining the first and second domains, and (c) is a second domain comprising an extracellular domain of a second transmembrane protein. In embodiments, the T cell is a T-cell progenitor cell. In embodiments, the T-cell or T-cell progenitor cell is obtained from a patient. In embodiments, the T-cell or T-cell progenitor cell is specific to a cancer antigen, (B) co-culturing the engineered T cell with a second cell, and (C) analyzing expression of a cytokine by the second cell. In embodiments, the engineered T cell is manufactured according to any of the embodiments disclosed herein. In embodiments, the second cell expresses a receptor for a transmembrane protein selected from 2B4, 4-1BB, ACVR1b, ACVR2A, ACVR2B, AXL, B7-H3, BCMA, BTLA BTNL3, BTNL8, BTNL3A1, BTNL3A2, CD2, CD27, CD30, CD31, CD31, CD40, CD48(SLAMF2), CD58(LFA3), CD137, CD160, CD200, CD226(PTAUDNAMI), CD244, CD247, CSF1R(CD115), CTLA-4, DcR3, Fas, FGFR3, Fn14, GITR, HVEM, ICOS, ICOSL, KIR2DL1, KIR2DL2, KIR2DL3, KIR3DL1, KIRDL2, LAG-3, LAP, LAYN, LTbR, NKG2A, NKp46(NCR1), NKp80(KLRF1), NTB-A, OX40, PD-1, PD-L1, PD-L2, PVR, RANK, SIGLEC7, SIGLEC9, SIRPα(CD172a), SLAMF6, TACI, TGFBR2, TIGIT, TIM-3, TMIGD2, TNFR1, TNFR2, TNFRSF25, TNFRSF4, TRAIL-R, VISTA, or VSIG8. In embodiments, the cytokine is selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA. In embodiments, the expression is analyzed by detection and/or quantitation of protein.

In embodiments, the expression is analyzed by detection and/or quantitation of mRNA (without limitation, e.g. using reverse transcriptase polymerase chain reaction). In embodiments, the expression is analyzed by detection and/or quantitation of protein (without limitation, e.g. using ELISA)

Pharmaceutical Composition

Populations of engineered T cells, as disclosed herein (e.g., comprising engineered T cells that express both chimeric proteins and, optionally, comprising additional engineered T cells that express one chimeric protein and/or T cells that express neither chimeric protein) may be used in the preparation of a pharmaceutical composition.

Aspects of the present invention include a pharmaceutical composition comprising a herein-disclosed engineered T cell as and a pharmaceutically acceptable excipient.

Another aspect is a pharmaceutical composition comprising a herein-disclosed population of engineered T cells and a pharmaceutically acceptable excipient.

In embodiments, at least one engineered T cell in the pharmaceutical composition is allogenic.

In embodiments, at least one engineered T cell in the pharmaceutical composition is autologous.

In embodiments, at least one engineered T cell in the pharmaceutical composition is allogenic and at least one engineered T cell in the pharmaceutical composition is autologous.

Any pharmaceutical composition comprising a herein described population of engineered T cells, may be supplemented with any herein described heterologous chimeric protein. The supplemented heterologous chimeric protein may be the same heterologous chimeric protein that is expressed and secreted by the engineered T cell and/or the supplemented heterologous chimeric protein may be different from the heterologous chimeric protein that is expressed and secreted by the engineered T cell.

Pharmaceutical compositions comprise a suitable amount of a pharmaceutically acceptable excipient so as to provide the form for proper administration. In embodiments, the pharmaceutically acceptable excipients are sterile when administered to a subject. Water is a useful excipient when a herein-disclosed composition is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any pharmaceutical composition can also comprise minor amounts of wetting or emulsifying agents, or pH buffering agents. In embodiments, pharmaceutical compositions may comprise a saline buffer (including, without limitation TBS, PBS, and the like). Examples of suitable pharmaceutical excipients are described in Remington's Pharmaceutical Sciences 1447-1676 (Alfonso R. Gennaro eds., 19th ed. 1995), incorporated herein by reference.

In embodiments, any herein-disclosed engineered T cell or composition comprising the same is formulated in accordance with routine procedures as a pharmaceutical composition adapted for a mode of administration disclosed herein.

Methods of Treatment

An aspect of the present invention is a method for treating a cancer in a subject in need thereof. The method comprises administering to the subject a therapeutically effective amount of a herein-disclosed pharmaceutical composition.

In embodiments, at least one engineered T cell in the pharmaceutical composition is allogenic.

In embodiments, at least one engineered T cell in the pharmaceutical composition is autologous.

In embodiments, at least one engineered T cell in the pharmaceutical composition is autologous and at least one engineered T cell in the pharmaceutical composition is allogenic.

In embodiments, the pharmaceutical composition induces the expression of a cytokine selected from IFNγ, TNFα, IL-2, IL-4, IL-5, IL-6, IL-7, IL-9, IL-10, IL-12, IL-13, IL-15, IL-21, IL-17A, IL-17F, IL-22, CXCL8, IL-1 alpha/IL-1F1, IL-1 beta/IL-1F2, LAP (TGF-beta 1), Lymphotoxin-alpha/TNF-beta, TGF-beta, TNF-alpha, TRANCE/TNFSF11/RANK L or a combination of any two or more thereof. In embodiments, the expression is analyzed by detection and/or quantitation of mRNA (without limitation, e.g. using reverse transcriptase polymerase chain reaction). In embodiments, the expression is analyzed by detection and/or quantitation of protein (without limitation, e.g. using ELISA).

In embodiments, the method treats a cancer selected from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers, or a combination thereof.

In embodiments, the cancer is a hematologic cancer selected from the group consisting of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia, or a combination thereof.

In embodiments, the method further comprises administering a pharmaceutical composition comprising any herein-disclosed heterologous chimeric protein. The heterologous chimeric protein may be the same heterologous chimeric protein that is expressed and secreted by the engineered T cell and/or the heterologous chimeric protein may be different from the heterologous chimeric protein that is expressed and secreted by the engineered T cell. In embodiments, the subject is administered the pharmaceutical composition comprising the heterologous chimeric protein before the subject is administered the pharmaceutical composition comprising the population of engineered T cells; the subject is administered the pharmaceutical composition comprising the heterologous chimeric protein after the subject is administered the pharmaceutical composition comprising the population of engineered T cells; or the subject is administered the pharmaceutical composition comprising the heterologous chimeric protein contemporaneous with administering the pharmaceutical composition comprising the population of engineered T cells. In embodiments, the dosage of the pharmaceutical composition comprising the heterologous chimeric protein is less than the dosage administered to a subject who has not or will not be administered the pharmaceutical composition comprising the population of engineered T cells.

In embodiments, the method further comprises administering to the subject a therapeutically-effective amount an immunosuppressive drug, e.g., which reduces or prevents Cytokine release syndrome. In embodiments, the immunosuppressive drug is an antibody directed against the interleukin-6 receptor (IL-6R), e.g., Tocilizumab. In embodiments, the immunosuppressive drug is administered before the pharmaceutical composition comprising the population of engineered T cells; the immunosuppressive drug is administered after the pharmaceutical composition comprising the population of engineered T cells; or the immunosuppressive drug is administered contemporaneous with the pharmaceutical composition comprising the population of engineered T cells.

In embodiments, the herein-disclosed pharmaceutical compositions can be administered via localized injection or localized infusion; via intravenous injection or intravenous infusion; via intra-arterial injection or intra-arterial infusion; or via intralymphatic injection or intralymphatic infusion.

In embodiments, the localized injection or localized infusion is directly to a tumor, i.e., intratumoral injection.

In embodiments, the localized injection or localized infusion is directly into an organ of the immune system, e.g., the bone marrow, thymus, spleen, adenoids, tonsils, lymph nodes, skin, and liver.

The number of engineered T cells in a pharmaceutical composition, i.e., the dosage of engineered T cells, administered can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific pharmaceutical composition being administered, the time of administration, the route of administration, the nature of the formulation, the particular disease being treated, the severity of the disorder, and the anatomical location of the disorder. Some variations in the dosage can be expected.

Aspects of the present invention further include use of a herein-disclosed engineered T cell in the manufacture of a medicament, e.g., a medicament for treatment of cancer.

Any aspect or embodiment disclosed herein can be combined with any other aspect or embodiment as disclosed herein.

The invention will be further described in the following example, which does not limit the scope of the invention described in the claims.

### EXAMPLES

The examples herein are provided to illustrate advantages and benefits of the present technology and to further assist a person of ordinary skill in the art with preparing or using the chimeric proteins of the present technology. The examples herein are also presented in order to more fully illustrate the preferred aspects of the present technology. The examples should in no way be construed as limiting the scope of the present disclosure, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1. Construction and Characterization of Illustrative Engineered T Cells

In the experiments of this example, separate plasmids encoding either pcDNA3.4-PD-1-Fc-OX40L or pcDNA3.4-CSF1R-Fc-CD40L were generated. Jurkat T-cells were then transfected separately with either pcDNA3.4-PD-1-Fc-OX40L plasmid DNA, or pcDNA3.4-CSF1R-Fc-CD40L plasmid DNA. The Jurkat T cells were then incubated for 48 hours prior to applying antibiotic selection (G418) to select for stably transfected cells over a two-week period. Single cell cloning of the stable pools enabled the selection of high heterologous chimeric protein-secreting clones to calculate the chimeric protein production level (pg/cell).

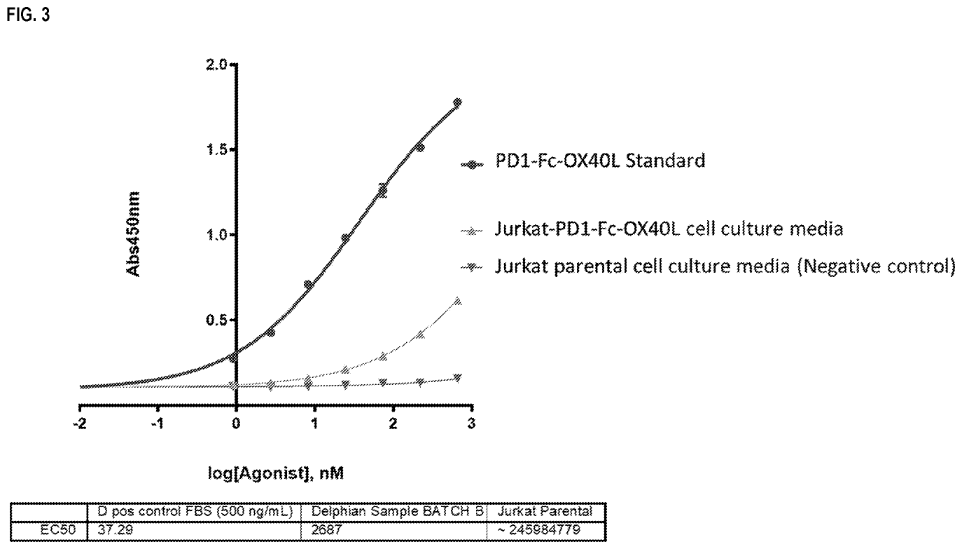

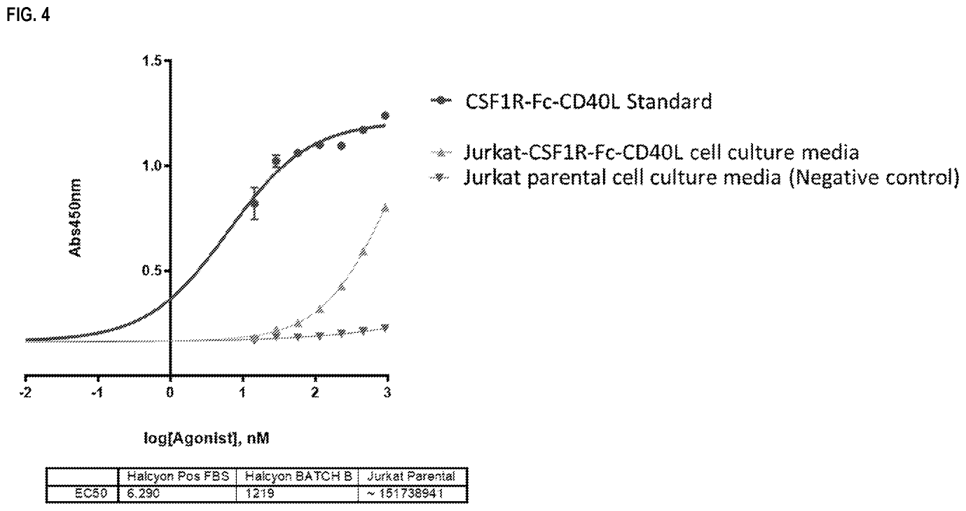

Following the emergence of G418-resistant stable pools for each construct after the two-week selection period, the engineered T cells were allowed to reach a high density and the engineered T cell culture media was then harvested, concentrated and tested for the presence of secreted PD-1-Fc-OX40L (FIG. 3), or secreted CSF1R-Fc-CD40L (FIG. 4) by a standard ELISA method. A concentrated Jurkat parental cell culture media was use as a negative control.

To detect the secretion of PD-1-Fc-OX40L or CSF1R-Fc-CD40L from engineered T cells in the cell culture media, a dual binding ELISA method confirmed the functionality of the secreted heterologous chimeric proteins. The ELISA method involved capturing the heterologous chimeric protein using a universal-heterologous chimeric protein specific anti-Fc antibody and detecting the heterologous chimeric proteins using either a biotinylated anti-human OX40L, in the case of PD-1-Fc-OX40L, or a biotinylated anti-CD40L antibody, in the case of CSF1R-Fc-CD40L. As shown in FIG. 3 and in FIG. 4, engineered T cells secreted high levels of PD-1-Fc-OX40L and CSF1R-Fc-CD40L.

### Example 2. The Ability of the Chimeric Proteins Secreted by T Cell Clones to Simultaneously Bind Two Ligands

An objective of this experiment was to understand whether the type I extracellular domain (ECD) and type II ECD present in the heterologous chimeric proteins secreted by T cell clones disclosed herein can simultaneously bind ligands. Towards that, dual binding assays were performed using the MESO SCALE DISCOVERY (MSD) platform.

Concentrated, de-salted culture supernatant was prepared as follows: 40 million Jurkat parental cells, Jurkat cells expressing human PD-1-Fc-4-1BBL chimeric protein (Jurkat/hPD-1-Fc-4-1BBL), Jurkat cells expressing human PD-1-Fc-OX40L chimeric protein (Jurkat/hPD-1-Fc-OX40L), or Jurkat cells expressing human CSF1R-Fc-CD40L chimeric protein (Jurkat/hCSF1R-Fc-CD40L) were cultured in 60 mL of media for 48 hours. After this, the media were harvested and concentrated 50-75× using AMICON-15 50K columns. Concentrated supernatant was desalted over a ZEBA desalting column or directly dialyzed in PBS and used in experiments. The resulting supernatant was also termed “neat” supernatant.

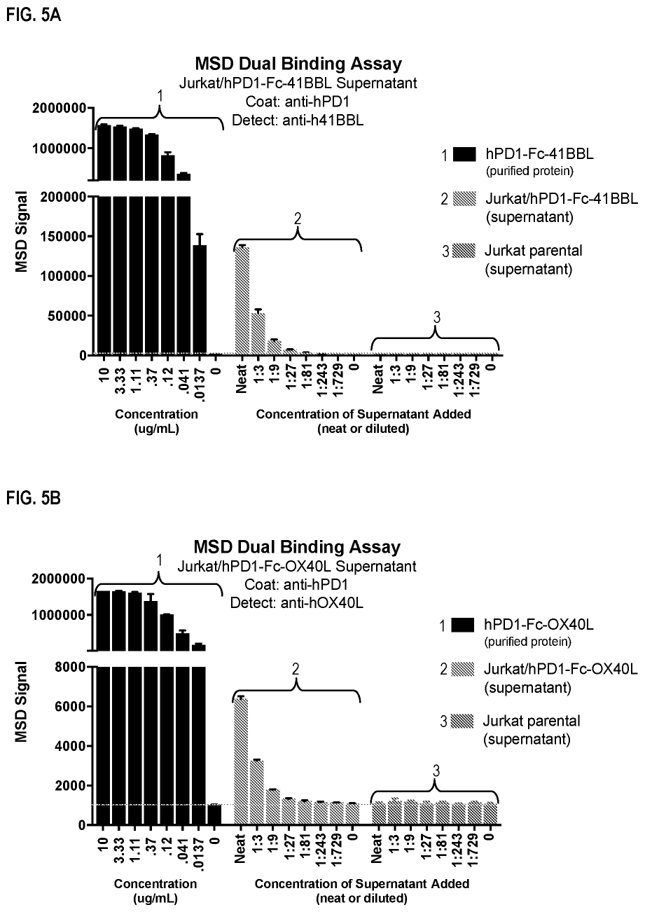

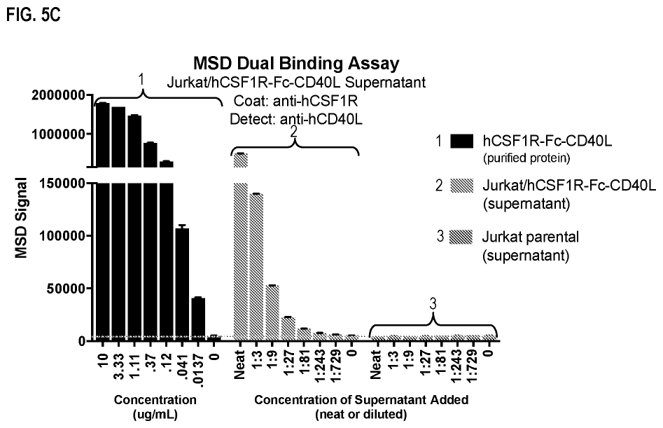

The PD-1-Fc-4-1BBL chimeric protein harbors the extracellular domain of PD-1 protein domain at or near the N-terminus and the extracellular domain of 4-1BB Ligand (4-1BBL) protein domain at or near the C-terminus. To understand whether the PD-1 and 4-1BBL can simultaneously bind to respective antibodies, an anti-human PD-1 antibody was coated on a plate. Three-fold serial dilutions neat culture supernatants of Jurkat parental cells or Jurkat/hPD-1-Fc-4-1BBL cells were added to the plate. As a positive control, 0, 0.0137, 0.041, 0.12, 0.37, 1.11, 3.33 and 10 μg/ml of the purified PD-1-Fc-4-1BBL chimeric protein was added. The plate was incubated to allow capture of any protein by the plate-bound anti-human PD-1 antibody. To detect capture of the PD-1-Fc-4-1BBL chimeric protein by the plate-bound anti-human PD-1 antibody, an anti-human 4-1BBL antibody was added. Generation of signal in this assay depends on bridging of the anti-human PD-1 and anti-human 4-1BBL antibodies by the PD-1-Fc-4-1BBL chimeric protein. Binding was detected using the MSD platform. As shown in FIG. 5A, The neat culture supernatant of the Jurkat parental cells showed only background signal. In contrast, the culture supernatant of Jurkat/hPD-1-Fc-4-1BBL cells produced a dose-dependent signal, indicating that the Jurkat/hPD-1-Fc-4-1BBL cells produced the hPD-1-Fc-4-1BBL chimeric protein that is capable of simultaneously binding the anti-human PD-1 and anti-human 4-1BBL antibodies (FIG. 5A). The purified hPD-1-Fc-4-1BBL chimeric protein produced a dose-dependent and saturable signal. Comparison of the signal produced by the purified hPD-1-Fc-4-1BBL chimeric protein with the signal produced by the culture supernatant of Jurkat/hPD-1-Fc-4-1BBL cells indicated that the neat culture supernatant contained a protein amount that was comparable to 0.0137 μg/ml of the purified PD-1-Fc-4-1BBL chimeric protein (FIG. 5A).

The PD-1-Fc-OX40L chimeric protein harbors the extracellular domain of PD-1 protein domain at or near the N-terminus and the extracellular domain of OX40 Ligand (OX40L) protein domain at or near the C-terminus. To understand whether the extracellular domains of PD-1 and OX40L can simultaneously bind to respective antibodies, an anti-human PD-1 antibody was coated on a plate. Three-fold serial dilutions neat culture supernatants of Jurkat parental cells or Jurkat/hPD-1-Fc-OX40L cells were added to the plate. As a positive control, 0, 0.0137, 0.041, 0.12, 0.37, 1.11, 3.33 and 10 μg/ml of the purified PD-1-Fc-OX40L chimeric protein was added. The plate was incubated to allow capture of any protein by the plate-bound anti-human PD-1 antibody. To detect capture of the PD-1-Fc-OX40L chimeric protein by the plate-bound anti-human PD-1 antibody, an anti-human OX40L antibody was added. Generation of signal in this assay depends on bridging of the anti-human PD-1 and anti-human OX40L antibodies by the PD-1-Fc-OX40L chimeric protein. Binding was detected using the MSD platform. As shown in FIG. 5B, The neat culture supernatant of the Jurkat parental cells showed only background signal that is capable of simultaneously binding the anti-human PD-1 and anti-human OX40L antibodies. In contrast, the culture supernatant of Jurkat/hPD-1-Fc-OX40L cells produced a dose-dependent signal, indicating that the Jurkat/hPD-1-Fc-OX40L cells produced the hPD-1-Fc-OX40L chimeric protein (FIG. 5B). The purified hPD-1-Fc-OX40L chimeric protein produced a dose-dependent and saturable signal. Comparison of the signal produced by the purified hPD-1-Fc-OX40L chimeric protein with the signal produced by the culture supernatant of Jurkat/hPD-1-Fc-OX40L cells indicated that the neat culture supernatant contained a protein amount that was slightly less than 0.0137 μg/ml of the purified PD-1-Fc-OX40L chimeric protein (FIG. 5B).

The CSF1R-Fc-CD40L chimeric protein harbors the extracellular domain of CSF1R protein domain at or near the N-terminus and the extracellular domain of CD40 Ligand (CD40L) protein domain at or near the C-terminus. To understand whether the extracellular domains of CSF1R and CD40L can simultaneously bind to respective antibodies, an anti-human CSF1R antibody was coated on a plate. Three-fold serial dilutions neat culture supernatants of Jurkat parental cells or Jurkat/hCSF1R-Fc-CD40L cells were added to the plate. As a positive control, 0, 0.0137, 0.041, 0.12, 0.37, 1.11, 3.33 and 10 μg/ml of the purified CSF1R-Fc-CD40L chimeric protein was added. The plate was incubated to allow capture of any protein by the plate-bound anti-human CSF1R antibody. To detect capture of the CSF1R-Fc-CD40L chimeric protein by the plate-bound anti-human CSF1R antibody, an anti-human CD40L antibody was added. Generation of signal in this assay depends on bridging of the anti-human CSF1R and anti-human CD40L antibodies by the CSF1R-Fc-CD40L chimeric protein. Binding was detected using the MSD platform. As shown in FIG. 5C, The neat culture supernatant of the Jurkat parental cells showed only background signal. In contrast, the culture supernatant of Jurkat/hCSF1R-Fc-CD40L cells produced a dose-dependent signal, indicating that the Jurkat/hCSF1R-Fc-CD40L cells produced the hCSF1R-Fc-CD40L chimeric protein that is capable of simultaneously binding the anti-human CSF1R and anti-human CD40L antibodies (FIG. 5C). The purified hCSF1R-Fc-CD40L chimeric protein produced a dose-dependent and saturable signal. Comparison of the signal produced by the purified hCSF1R-Fc-CD40L chimeric protein with the signal produced by the culture supernatant of Jurkat/hCSF1R-Fc-CD40L cells indicated that the neat culture supernatant contained a protein amount that was comparable to 0.12 μg/ml of the purified CSF1R-Fc-CD40L chimeric protein (FIG. 5C).

These data demonstrate that the T cells disclosed herein expressed and secreted the chimeric proteins that are capable of simultaneously binding and bridging the ligands via the two extracellular domains.

### Example 3. Construction of Cell Lines Expressing Ligands of the Extracellular Domains

To study whether the extracellular domains present in the chimeric proteins expressed by the Jurkat/hPD-1-Fc-4-1BBL, Jurkat/hPD-1-Fc-OX40L, and Jurkat/hCSF1R-Fc-CD40L cells bind cells expressing their cognate ligands model cell lines were constructed. Specifically, CHO-K1 or HT1080 cells were engineered to over-express certain ligands on their surface.

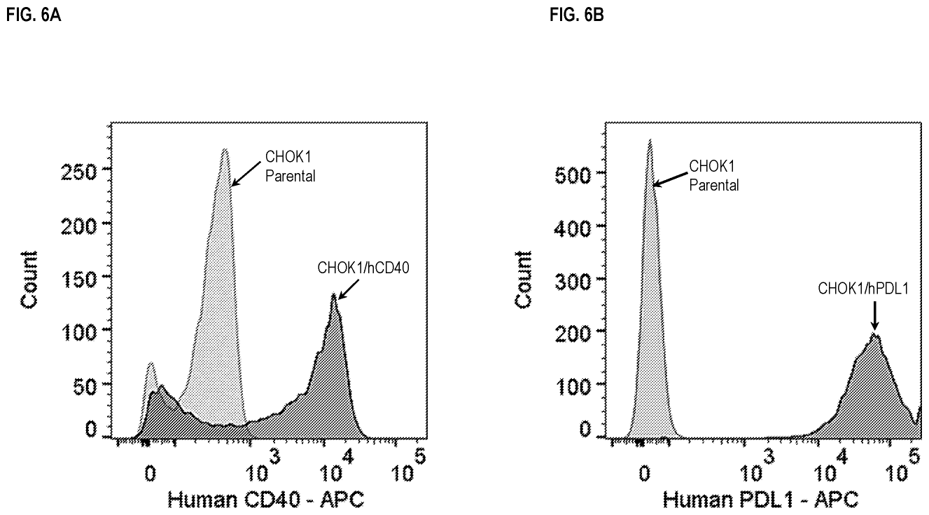

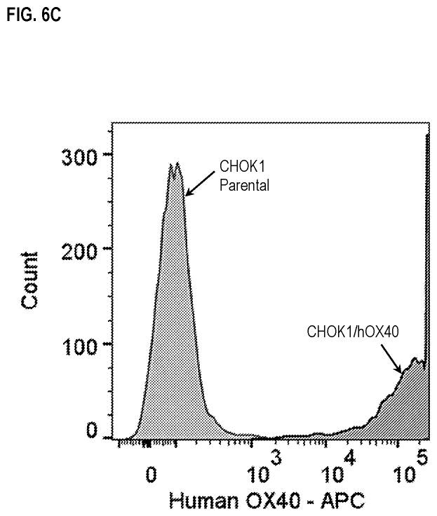

CHO-K1 parental cells stably expressing human CD40 were constructed. Cells of a CD40 positive clone were called the CHO-K1/hCD40 cells. The CHO-K1/hCD40 cells and parental CHO-K1 cells were incubated with an anti-CD40 antibody. Binding was detected using flow cytometry. As shown in FIG. 6A, the anti-CD40 antibody stained the CHO-K1/hCD40 cells but not to the parental CHO-K1 cells. These data indicate successful construction of a cell line stably expressing human CD40.

CHO-K1 parental cells stably expressing human PD-L1 were constructed. Cells of a PD-L1 positive done were called the CHO-K1/hPD-L1 cells. The CHO-K1/hPD-L1 cells and parental CHO-K1 cells were incubated with an anti-PD-L1 antibody. Binding was detected using flow cytometry. As shown in FIG. 6B, the anti-PD-L1 antibody stained the CHO-K1/hPD-L1 cells but not to the parental CHO-K1 cells. These data indicate successful construction of a cell line stably expressing human PD-L1.

CHO-K1 parental cells stably expressing human OX40 were constructed. Cells of a OX40 positive clone were called the CHO-K1/hOX40 cells. The CHO-K1/hOX40 cells and parental CHO-K1 cells were incubated with an anti-OX40 antibody. Binding was detected using flow cytometry. As shown in FIG. 5C, the anti-OX40 antibody stained the CHO-K1/hOX40 cells but not to the parental CHO-K1 cells. These data indicate successful construction of a cell line stably expressing human OX40.

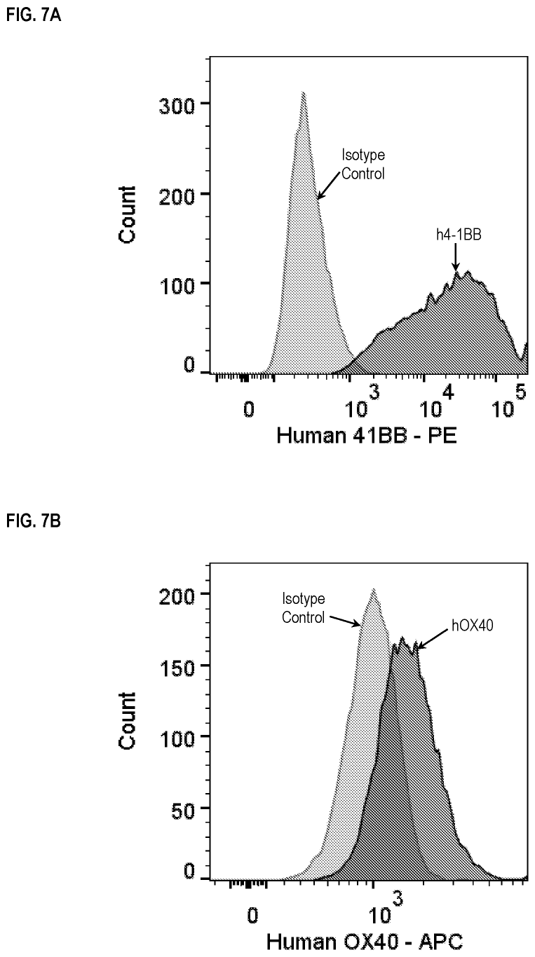

HT1080 parental cells stably expressing human 4-1BB were constructed. Cells of a 4-1BB positive clone were called the HT1080/h4-1BB cells. The HT1080/h4-1BB cells and parental HT1080 cells were incubated with an anti-4-1BB antibody. Binding was detected using flow cytometry. As shown in FIG. 7A, the anti-4-1BB antibody stained the HT1080/h4-1BB cells but not to the parental HT1080 cells. These data indicate successful construction of a cell line stably expressing human 4-1BB. The HT1080 express other TNF receptors (e.g. hOX40) at moderate levels. To confirm this, The HT1080/h4-1BB cells and parental HT1080 cells were incubated with an anti-OX40 antibody. Binding was detected using flow cytometry. As shown in FIG. 7B, the anti-OX40 antibody stained both parental HT1080 cells and the HT1080/h4-1BB cells.

### Example 4. Binding of the Chimeric Proteins Secreted by T Cell Clones Disclosed Herein to Cells Expressing Ligands of the Extracellular Domains

An objective of this experiment was to evaluate the binding of both ECDs present the chimeric proteins secreted by T cell clones disclosed herein to their respective cognate ligands. Towards that objective the extent binding to cells expressing ligands of each ECD was separately quantitated.

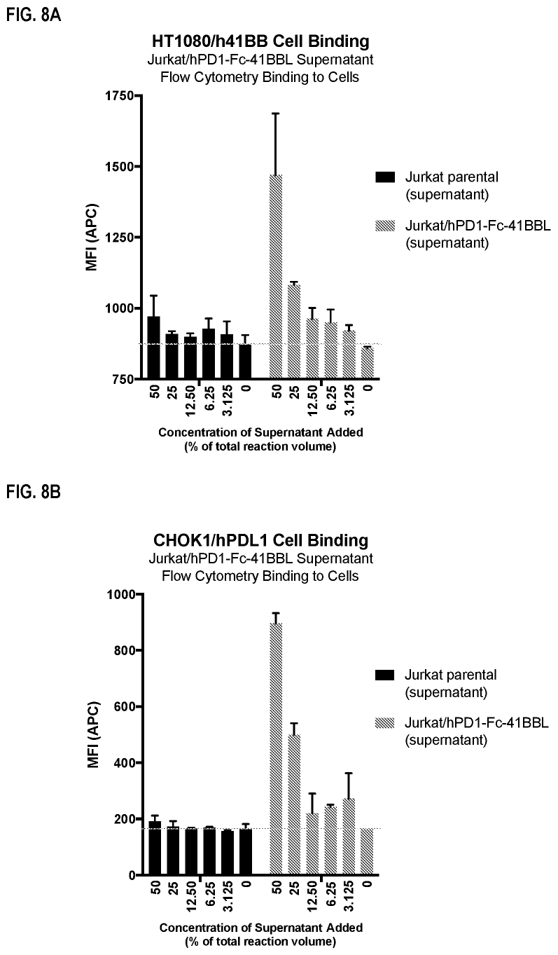

In one experiment, the HT1080/h4-1BB cells were incubated with increasing amounts of the neat culture supernatant of Jurkat parental or the Jurkat/hPD-1-Fc-4-1BBL cells produced as discussed in Example 2. Binding was detected using flow cytometry and the mean fluorescence intensity (MFI) was calculated and plotted as a function of the amount of the neat culture supernatant of Jurkat parental or Jurkat/hPD-1-Fc-4-1BBL cells. As shown in FIG. 8A, the neat culture supernatant of the Jurkat/hPD-1-Fc-4-1BBL cells showed dose-dependent binding to the HT1080/h4-1BB cells. In contrast, the neat culture supernatant of the Jurkat parental cells showed only background signal (FIG. 8A). Further, the CHO-K1/hPD-L1 cells were incubated with increasing amounts of the neat culture supernatant of the Jurkat parental or Jurkat/hPD-1-Fc-4-1BBL cells produced as discussed in Example 2. Binding was detected using flow cytometry and the MFI was calculated and plotted as a function of the amount of the neat culture supernatant of Jurkat parental or Jurkat/hPD-1-Fc-4-1BBL cell. As shown in FIG. 8B, the neat culture supernatant of the Jurkat/hPD-1-Fc4-1BBL cells showed dose-dependent binding to the CHO-K1/hPD-L1 cells. In contrast, the neat culture supernatant of the Jurkat parental cells showed only background signal (FIG. 8B). These data indicate that the Jurkat/hPD-1-Fc-4-1BBL cells produced the hPD-1-Fc-4-1BBL heterologous chimeric protein that is capable of specifically binding to cells expressing human 4-1BB, which is the cognate ligand of 4-1BBL, and to cells expressing human PD-1, which is the cognate ligand of PD-1.

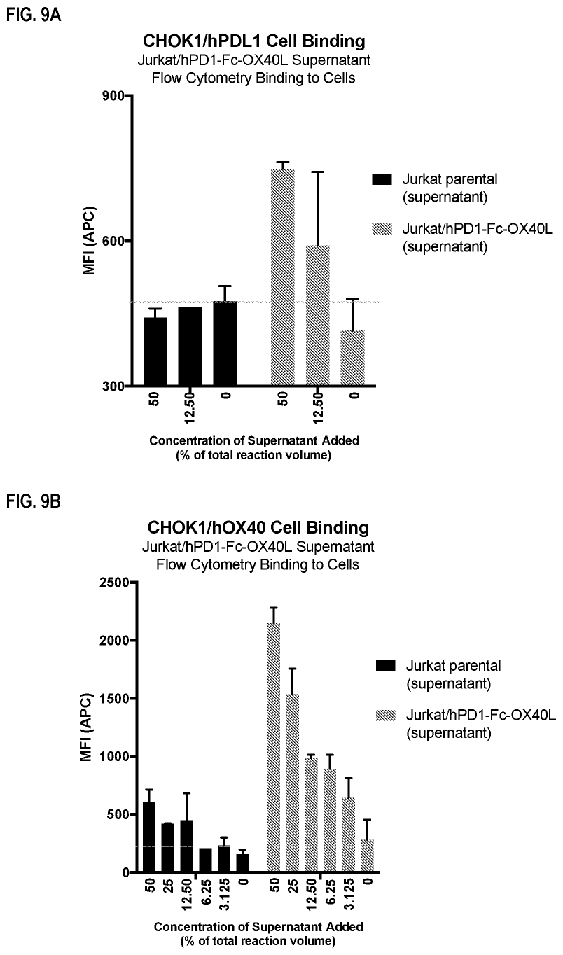

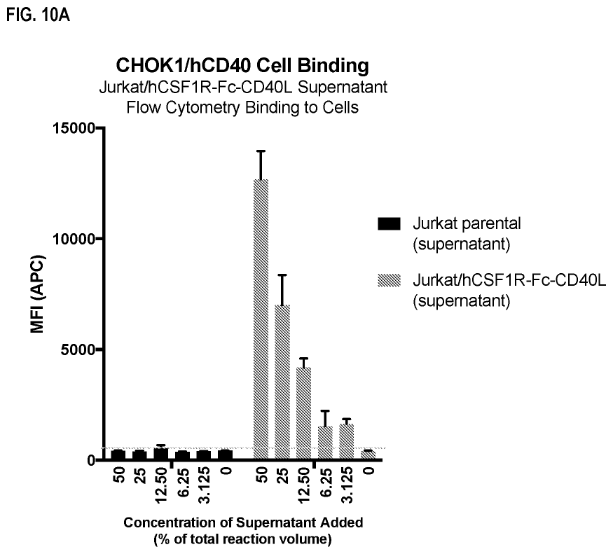

In another experiment, the CHO-K1/hPD-L1 cells were incubated with increasing amounts of the neat culture supernatant of the Jurkat parental or Jurkat/hPD-1-Fc-OX40L cells produced as discussed in Example 2. Binding was detected using flow cytometry and the MFI was calculated and plotted as a function of the amount of the neat culture supernatant of Jurkat parental or Jurkat/hPD-1-Fc-OX40L cells. As shown in FIG. 9A, the neat culture supernatant of the Jurkat/hPD-1-Fc-OX40L cells showed dose-dependent binding to the CHO-K1/hPD-L1 cells. In contrast, the neat culture supernatant of the Jurkat parental cells showed only background signal (FIG. 9A). Further, the CHO-K1/hOX40 cells were incubated with increasing amounts of the neat culture supernatant of the Jurkat parental or Jurkat/hPD-1-Fc-OX40L cells produced as discussed in Example 2. Binding was detected using flow cytometry and the MFI was calculated and plotted as a function of the amount of the neat culture supernatant of Jurkat parental or Jurkat/hPD-1-Fc-OX40L cells. As shown in FIG. 9B, the neat culture supernatant of the Jurkat/hPD-1-Fc-OX40L cells showed dose-dependent binding to the CHO-K1/hOX40 cells. In contrast, the neat culture supernatant of the Jurkat parental cells showed only background signal (FIG. 9B). These data indicate that the Jurkat/hPD-1-Fc-OX40L cells produced the hPD-1-Fc-OX40L heterologous chimeric protein that is capable of specifically binding to cells expressing human OX40, which is the cognate ligand of OX40L, and to cells expressing human PD-L1, which is the cognate ligand of PD-1.

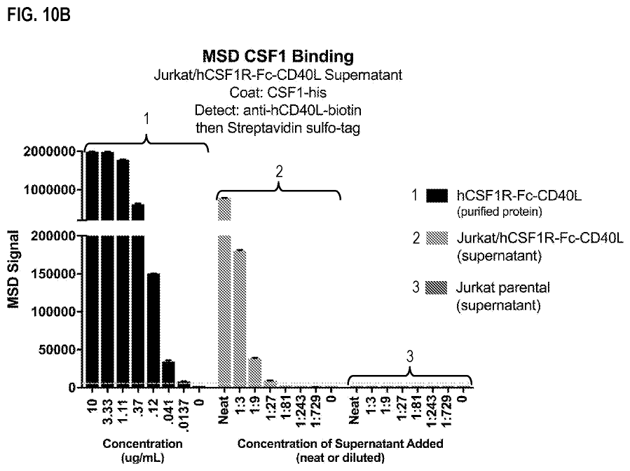

In yet another experiment, the CHO-K1/hCD40 cells were incubated with increasing amounts of the neat culture supernatant of the Jurkat parental or Jurkat/hCSF1R-Fc-CD40L cells produced as discussed in Example 2. Binding was detected using flow cytometry and the MF was calculated and plotted as a function of the amount of the neat culture supernatant of Jurkat parental or Jurkat/hCSF1R-Fc-CD40L cells. As shown in FIG. 10A, the neat culture supernatant of the Jurkat/hCSF1R-Fc-CD40L cells showed dose-dependent binding to the CHO-K1/hCD40 cells. In contrast, the neat culture supernatant of the Jurkat parental cells showed only background signal (FIG. 10A).

To detect binding of the chimeric protein produced by Jurkat/hCSF1R-Fc-CD40L cells to human CSF1, which is a soluble protein, the MSD platform was used. Briefly, recombinant human CSF1 protein was coated on MSD plates. Increasing amounts of the neat culture supernatant of the Jurkat parental or Jurkat/hCSF1R-Fc-CD40L cells produced as discussed in Example 2 were added. The plates were incubated to allow capture of any CSF-1 binding protein by the plate-bound CSF1. To detect capture of the hCSF1R-Fc-CD40L chimeric protein by the plate-bound CSF1, anti-human CD40L-biotin antibody was added. The MSD streptavidin reagent was added for detection. The MSD signal was plotted as a function of the amount of the neat culture supernatant of Jurkat parental or Jurkat/hCSF1R-Fc-CD40L cells. As shown in FIG. 10B, the neat culture supernatant of the Jurkat/hCSF1R-Fc-CD40L cells showed dose-dependent binding to human CSF1. In contrast, the neat culture supernatant of the Jurkat parental cells showed only background signal (FIG. 10B). The purified hCSF1R-Fc-CD40L chimeric protein produced a dose-dependent and saturable signal. Comparison of the signal produced by the purified hCSF1R-Fc-CD40L chimeric protein with the signal produced by the culture supernatant of Jurkat/hCSF1R-Fc-CD40L cells indicated that the neat culture supernatant contained a protein amount that was comparable to 0.37 μg/ml of the purified CSF1R-Fc-CD40L chimeric protein (FIG. 5C).

These data indicate that the Jurkat/hCSF1R-Fc-CD40L cells produced the hCSF1R-Fc-CD40L heterologous chimeric protein that is capable of specifically binding to cells expressing human CD40, which is the cognate ligand of CD40L, and to plate-bound human CSF1, which is the cognate ligand of CSF1R.

Collectively, these data demonstrate that the T cells disclosed herein expressed and secreted functionally active chimeric proteins that were capable of binding to cells expressing two types of cognate ligands. Thus, the chimeric proteins would bridge two cells types (e.g. cancer cells and immune cells) when the two ligands are expressed by the two cell types.

### Example 5. Cytokine Induction by the Chimeric Proteins Secreted by T cell Clones Disclosed Herein

An objective of this experiment was to understand whether the binding of the chimeric proteins secreted by T cell clones disclosed herein to their cognate ligands produces immunological effects. Towards that objective the induction of cytokines was evaluated in comparison with house-keeping control genes.

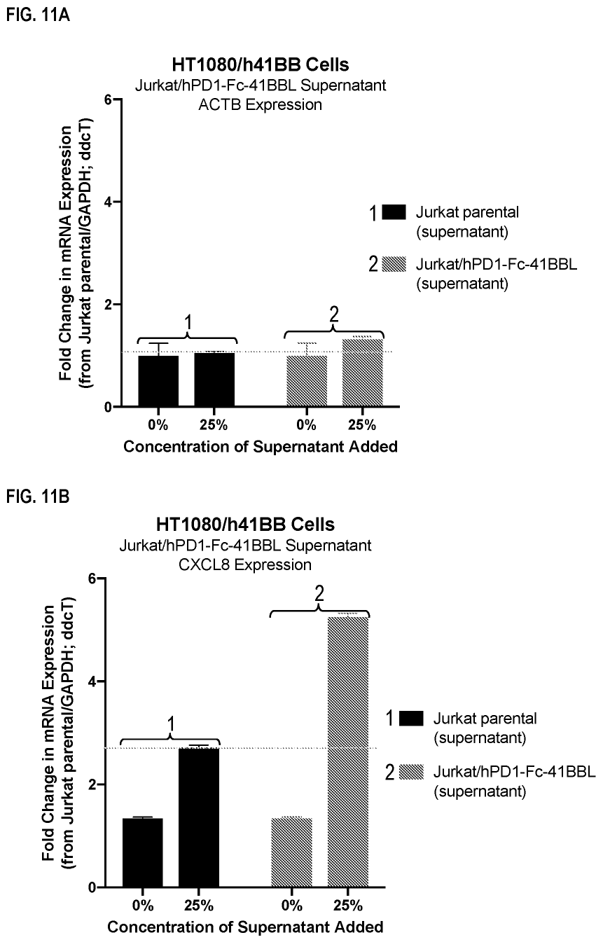

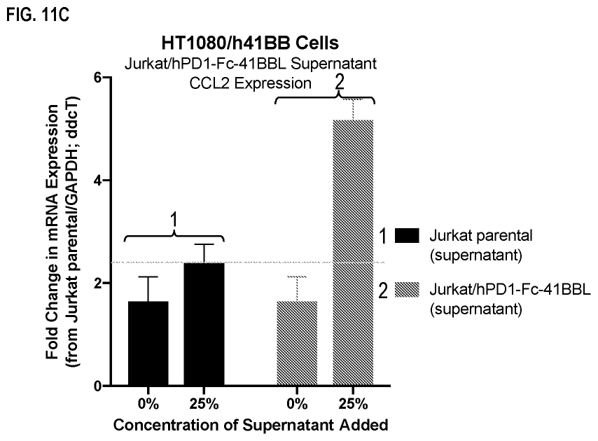

In one experiment, 250,000 HT1080/h4-1BB cells were grown overnight. The following day, 0% or 25% (of total cell culture volume) of the neat culture supernatant of Jurkat parental cells or the Jurkat/hPD1-Fc-4-1BBL cells, which was generated as described in Example 2, was added to the cells. Incubation was continued for an additional 3 hours. After 3 hours, RNA was isolated from the HT10801h4-1BB cells, cDNA was synthesized, and gene expression of GAPDH, ACTB (a house-keeping control gene), CXCL8, and CCL2 was assessed by qRT-PCR. GAPDH was used to normalize gene expression and fold-induction according to the delta-delta CT method. As shown in FIG. 11A, the addition of the neat culture supernatant of Jurkat parental cells or the Jurkat/hPD1-Fc-4-1BBL cells did not appreciably affect the expression of ACTB gene expression. The addition of the neat culture supernatant of Jurkat parental cells caused a slight inducement of CXCL8 and CCL2 genes (FIG. 11B and FIG. 11C). This may be explained by the factors secreted by Jurkat cells. In contrast, as shown in FIG. 11 and FIG. 11C, the neat culture supernatant of Jurkat/hPD1-Fc-4-1BBL cells induced the expression of CXCL8 (FIG. 11B) and CCL2 (FIG. 11C) by several fold. The induction of CXCL8 (FIG. 11B) (p<0.0001) was statistically significant by unpaired t-test with Welch's correction. Similarly, the induction of CCL2 (FIG. 11C) (p<0.001) was statistically significant by unpaired t-test with Welch's correction. These data indicate that the Jurkat/hPD-1-Fc-4-1BBL cells produced the hPD-1-Fc-4-1BBL heterologous chimeric protein that is capable of specifically inducing signaling downstream to 4-1BB.

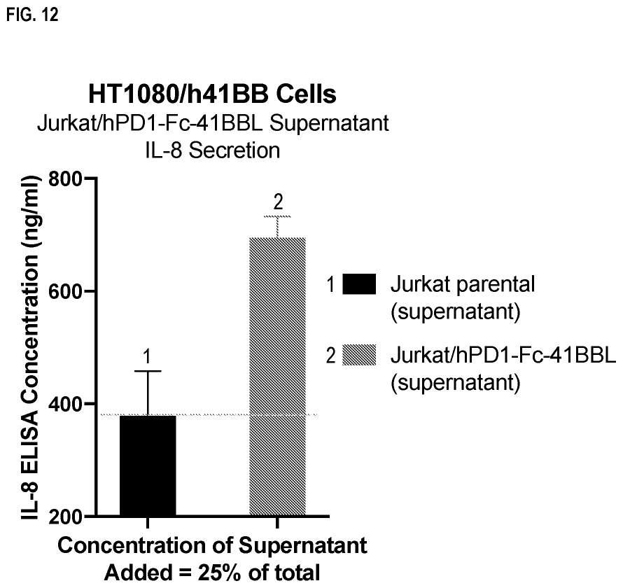

To understand whether the inducing signaling downstream to 4-1BB actually culminates in cytokine production, IL-8 production by HT1080/h4-1BB cells was measured. 250,000 HT1080/h4-1BB cells were grown overnight. The following day, 25% (of total cell culture volume) of the neat culture supernatant of Jurkat parental cells or the Jurkat/hPD1-Fc-4-1BBL cells, which was generated as described in Example 2, was added to the cells. Incubation was continued for an additional 3 hours. After 3 hours, media were removed from the HT1080/h4-1BB cell culture and IL-8 was assessed by ELISA. As shown in FIG. 12, the neat culture supernatant of the Jurkat/hPD1-Fc-4-1BBL cells, caused induction of induction of IL-8, compared to the neat culture supernatant of Jurkat parental cells (p=0.0696 by unpaired t-test with Welch's correction). These data indicate that the Jurkat/hPD-1-Fc-4-1BBL cells produced the hPD-1-Fc-4-1BBL heterologous chimeric protein that is capable of inducing cytokine expression.

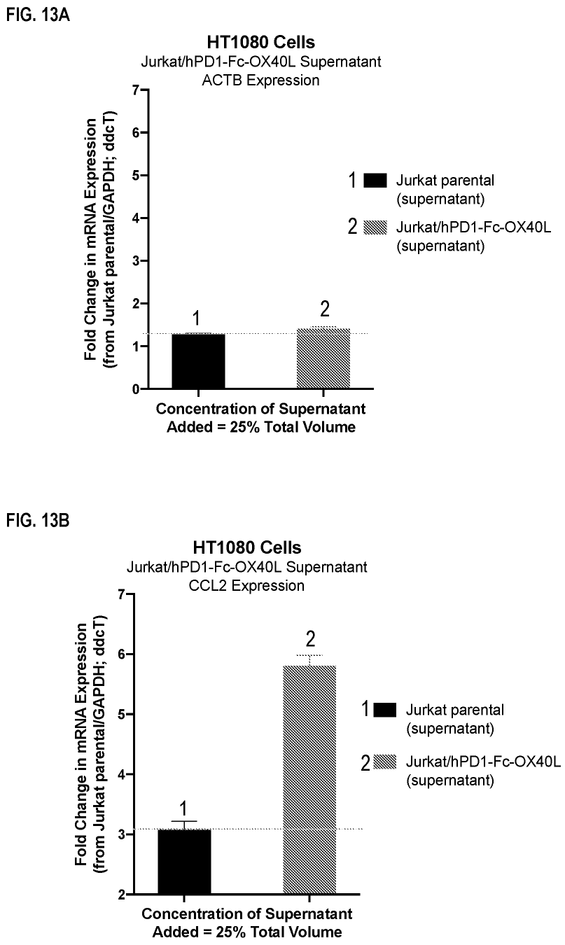

In another experiment, 250,000 HT1080 cells were grown overnight. The following day, 25% (of total cell culture volume) of the neat culture supernatant of Jurkat parental cells or the Jurkat/hPD1-Fc-OX40L cells, which was generated as described in Example 2, was added to the cells. Incubation was continued for an additional 3 hours. After 3 hours, RNA was isolated from the HT1080 cells, cDNA was synthesized, and gene expression of GAPDH, ACTB, CCL2 was assessed by qRT-PCR. GAPDH was used to normalize gene expression and fold-induction according to the delta-delta CT method. The addition of the neat culture supernatant of Jurkat/hPD1-Fc-OX40L cells did not appreciably affect the expression of ACTB gene expression compared to the neat culture supernatant of Jurkat parental cells (FIG. 13A). However, as shown in FIG. 13B, the neat culture supernatant of Jurkat/hPD1-Fc-OX40L cells induced the expression of CCL2 compared to the neat culture supernatant of Jurkat parental cells. The induction of CCL2 (FIG. 11B) (p<0.0001) was statistically significant by unpaired t-test with Welch's correction. These data indicate that the Jurkat/hPD-1-Fc-OX40L cells produced the hPD-1-Fc-OX40L heterologous chimeric protein that is capable of specifically inducing signaling downstream to OX40.

Collectively, these data indicate that the Jurkat/hPD-1-Fc-4-1BBL and Jurkat/hPD1-Fc-OX40L cells produced the hPD-1-Fc-4-1BBL and hPD1-Fc-OX40L heterologous chimeric proteins, respectively, that is capable of specifically binding to cells expressing human 4-1BB or OX40, the cognate ligands of 4-1BBL and OX40L, and induce signaling downstream to 4-1BB or OX40, which culminates in the induction of cytokines.

### Example 6. Induction of Downstream Signaling by the T Cell Clones Disclosed Herein in Sensitive Cells During Co-Culture

An objective of this experiment was to more closely mimic the in vivo interaction of T cells disclosed herein and the cells that are sensitive to the chimeric proteins secreted by T cell clones. Towards that objective, the PATHHUNTER U2OS-NIK/NFκB reporter cells (DISCOVERX), which are commercially available genetically engineered human bone osteosarcoma epithelial cells, were used. These cells enable assessment of ligand (e.g. CD40L) induced NF-κB signaling, which can be detected in form of a bioluminescence signal.

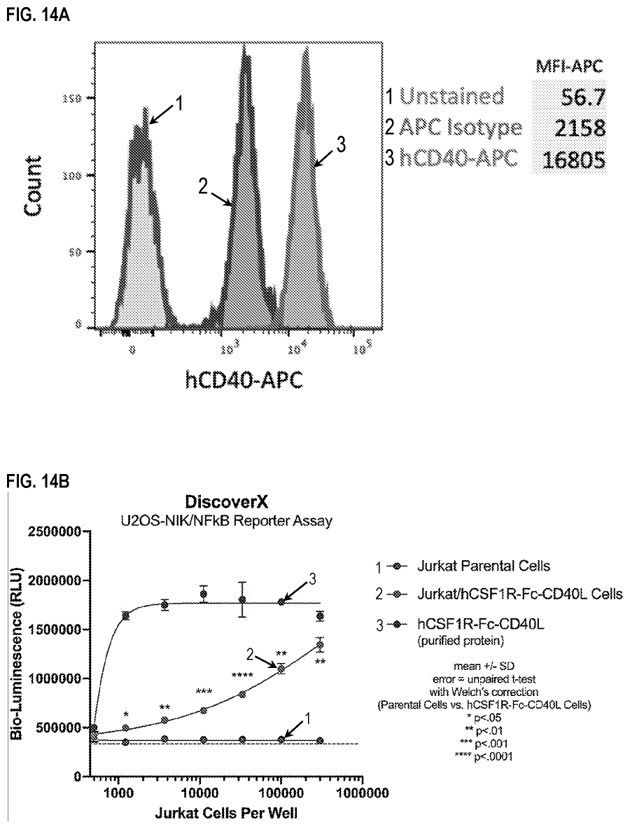

To demonstrate that the PATHHUNTER U2OS-NIK/NFκB reporter cells express CD40, those cells were stained using APC-conjugated isotype antibody or an anti-human CD40-APC antibody, and analyzed by flow cytometry in comparison with unstained cells. As shown in FIG. 14A, the PATHHUNTER U2OS-NIK/NFκB reporter cells were stained by the anti-human CD40-APC antibody, indicating that the PATHHUNTER U2OS-NIK/NFκB reporter cells express human CD40 (hCD40).

To understand whether the Jurkat/hCSF1R-Fc-CD40L cells induce the PATHHUNTER U2OS-NIK/NFκB reporter cells in co-culture, 50,000 PATHHUNTER U2OS-NIK/NFκB reporter cells were cultured overnight according to the manufacturer's recommendations. The following day, increasing numbers of Jurkat parental cells or Jurkat/hCSF1R-Fc-CD40L cells were co-cultured with the PATHHUNTER U2OS-NIK/NFκB reporter cells. Specifically, 0, about 500, about 1,230, about 3,700, about 11,100, about 33,300, about 100,000, or about 300,000, Jurkat parental cells or Jurkat/hCSF1R-Fc-CD40L cells were co-cultured with the PATHHUNTER U2OS-NIK/NFκB reporter cells. 3-fold serial dilutions of 15 μg/mL purified CSF1R-Fc-CD40L chimeric protein were used as a positive controls. After 6 hours in culture, the DISCOVERX luminescence reagent was added, and bioluminescence was determined using a PROMEGA GLOMAX instrument. As shown in FIG. 14B, the Jurkat/hCSF1R-Fc-CD40L cells also induced bioluminescence, indicating the induction of CD40L-induced NF-κB signaling. The purified CSF1R-Fc-CD40L chimeric protein, the positive control, induced bioluminescence (FIG. 14B), and Jurkat parental cells did not induce such bioluminescence (FIG. 14B). At the higher Jurkat/hCSF1R-Fc-CD40L cell numbers, the bioluminescence signal approached the maximal induction produced by the purified hCSF1R-Fc-CD40L chimeric protein. As shown in FIG. 14B, the induction of NFκB signaling by Jurkat/hCSF1R-Fc-CD40L cells was statistically significant every dose tested. These results demonstrate that during the co-culture, Jurkat/hCSF1R-Fc-CD40L produced the hCSF1R-Fc-CD40L protein, which bound to CD40 expressed by the PATHHUNTER U2OS-NIK/NFκB reporter cells and induced downstream NFκB signaling in the PATHHUNTER U2OS-NIK/NFκB reporter cells.

These data indicate that the Jurkat/hCSF1R-Fc-CD40L cells produced the hCSF1R-Fc-CD40L heterologous chimeric protein that is capable of specifically binding to cells expressing human CD40, the cognate ligands of CD40L, and inducing signaling downstream to CD40, which culminates in the induction of cytokines.

### Example 7. Induction of Cytokines by the T Cell Clones Disclosed Herein in Cells Expressing ECD Ligands During Co-Culture

An objective of this experiment was to explore whether the chimeric proteins secreted by T cell clones disclosed produce immunological effects. Such immunological effects might arise from binding of the chimeric proteins to their ligands and/or binding of the chimeric antigen receptor to the antigen against which they are specific. Towards that objective the induction of cytokines was evaluated in comparison with house-keeping control genes in co-culture experiments.

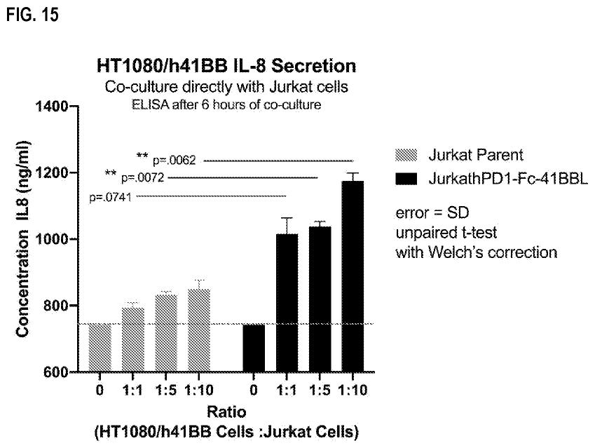

In one experiment, 250,000 HT1080/h4-1BB cells were grown overnight. The following day, Jurkat/hPD1-Fc-4-1BBL cells were plated on top of the HT1080/h4-1BB cells at HT1080/h4-1BB cell: Jurkat cell ratio of 1:0 (control without Jurkat cells), 1:1, 1:5 and 1:10. Incubation was continued for an additional 6 hours. After 6 hours, media were removed from the co-culture and IL-8 was assessed by ELISA. As shown in FIG. 15, the Jurkat/hPD1-Fc-4-1BBL cells caused induction of induction of IL-8, compared to Jurkat parental cells (p=0.0696 by unpaired t-test with Welch's correction). Further, as shown in FIG. 15, the induction of IL-8 secretion was statistically significant. These data indicate that the Jurkat/hPD-1-Fc-4-1BBL cells produced the hPD-1-Fc-4-1BBL heterologous chimeric protein that is capable of inducing cytokine expression.

Collectively, these data indicate that the T cell clones disclosed herein produce the heterologous chimeric proteins that are capable of specifically binding to cells expressing the ligands/receptors of the chimeric proteins, and inducing the signaling downstream to the ligands/receptors, which culminates in the induction of cytokines.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. An engineered T cell that expresses:
a chimeric antigen receptor comprising an antigen-binding domain, a transmembrane domain, and an intracellular domain which comprises a costimulatory domain and/or a signaling domain; and
a heterologous chimeric protein comprising the general structure:
N terminus-(a)-(b)-(c)-C terminus,
wherein:
(a) is a first domain comprising an extracellular domain of a first transmembrane protein,
(b) is al inker domain adjoining the first and second domains, and
(c) is a second domain comprising an extracellular domain of a second transmembrane protein.

2. The engineered T cell of claim 1, wherein the first domain comprises substantially the entire extracellular domain of the first transmembrane protein and/or the second domain comprises substantially the entire extracellular domain of the second transmembrane protein.

3. The engineered T cell of claim 1 or claim 2, wherein the first domain is capable of binding a ligand/receptor of the first transmembrane protein and the second domain is capable of binding a ligand/receptor of the second transmembrane protein.

4. The engineered T cell of any one of claims 1 to 3, wherein the first domain is capable of inhibiting an immunosuppressive signal when bound to its ligand/receptor and/or the second domain is capable of activating an immune stimulatory signal when bound to its ligand/receptor.

5. The engineered T cell of any one of claims 1 to 4, wherein the ligand/receptor of the first transmembrane protein is expressed by a cancer cell and/or the ligand/receptor of the second transmembrane protein is expressed by the engineered T cell and/or a native T cell.

6. The engineered T cell of any one of claims 1 to 4, wherein the antigen-binding domain is capable of binding an antigen expressed by a cancer cell.

7. The engineered T cell of any of claims 1 to 4, wherein the heterologous chimeric protein is secreted in response to activation of the chimeric antigen receptor expressed by the engineered T cell, e.g., wherein activation of the chimeric antigen receptor stimulates a nuclear factor of activated T cells (NFAT) promoter.

8. The engineered T cell of any of claims 1 to 4, wherein the engineered T cell expresses an alpha/beta T cell receptor.

9. The engineered T cell of any of claims 1 to 4, wherein the engineered T cell expresses a gamma/delta T cell receptor.

10. The engineered T cell of any one of claims 5 to 9, wherein:
the heterologous chimeric protein is capable of forming a stable synapse between the cancer cell and the engineered T cell when its first domain is bound to the ligand/receptor of the first transmembrane protein expressed by the cancer cell and the second domain is bound to the ligand/receptor of the second transmembrane protein expressed by the engineered T cell; and/or
the chimeric antigen receptor is capable of forming a stable synapse between the cancer cell and the engineered T cell when bound to the antigen expressed by the cancer cell.

11. The engineered T cell of claim 10, wherein the stable synapse provides a spatial orientation that favors an anti-cancer effect/tumor reduction by the engineered T cell.

12. The engineered T cell of claim 11, wherein the spatial orientation positions the engineered T cell to attack the cancer cell.

13. The engineered T cell of any one of claims 10 to 12, wherein the stable synapse allows for proliferation of the engineered T cell near the cancer cell.

14. The engineered T cell of any of claims 10 to 13, wherein the stable synapse allows for recruitment and proliferation of non-engineered, native, T cells near the cancer cells/the tumor site.

15. The engineered T cell of any one of claims 10 to 14, wherein the stable synapse allows sufficient signal transmission to provide expression and/or release of a stimulatory signal.

16. The engineered T cell of claim 15, wherein the stimulatory signal is a cytokine.

17. The engineered T cell of any one of claims 1 to 16, wherein the heterologous chimeric protein and/or chimeric antigen receptor is capable of increasing or preventing a decrease in a sub-population of CD4+ and/or CD8+ T cells.

18. The engineered T cell of any one of claims 1 to 17, wherein the heterologous chimeric protein increases the number of non-engineered T cell clones present near the cancer cells/the tumor site.

19. The engineered T cell of any one of claims 1 to 18, wherein the heterologous chimeric protein is secreted by the engineered T cell.

20. The engineered T cell of any one of claims 1 to 19, wherein the linker domain comprises at least one cysteine residue capable of forming a disulfide bond and/or comprises a hinge-CH2-CH3 Fc domain.

21. The engineered T cell of claim 20, wherein the hinge-CH2-CH3 Fc domain is derived from IgG4, e.g., human IgG4.

22. The engineered T cell of claim 20 or claim 21, wherein the linker domain comprises an amino acid sequence that is at least 95% identical to the amino acid sequence of SEQ ID NO: 1, SEQ ID NO: 2, or SEQ ID NO: 3.

23. The engineered T cell of any one of claims 1 to 22, wherein the first transmembrane protein is selected from the group consisting of PD-1, CSF1R, TIM-3, BTLA, CTLA-4, LAG-3, B7-H3, TMIGD2, TIGIT, CD172a/SIRPα, BTNL3, BTNL8, BTNL3A1, BTNL3A2, and VSIG8.

24. The engineered T cell of any one of claims 1 to 23, wherein the second transmembrane protein is selected from the group consisting of OX40 ligand (OX40L), CD40 ligand (CD40L), 4-1BB ligand (4-1BBL), GITR ligand (GITRL), LIGHT (CD258), CD70, CD30 ligand, TRAIL, and T1A.

25. The engineered T cell of claim 24, wherein the first transmembrane protein is PD-1.

26. The engineered T cell of claim 25, wherein the second transmembrane protein is OX40L.

27. The engineered T cell of claim 25, wherein the second transmembrane protein is 4-1BBL.

28. The engineered T cell of claim 24, wherein the first transmembrane protein is CSF1R.

29. The engineered T cell of claim 28, wherein the second transmembrane protein is CD40L.

30. The engineered T cell of any one of claims 1 to 29, wherein the chimeric antigen receptor is encoded by a nucleic acid introduced into a genomic safe harbor (GSR) in the engineered T cell's genome.

31. The engineered T cell of claim 30, wherein the GSR is the adeno-associated virus site 1 (AAVS1); the chemokine (C-C motif) receptor 5 (CCR5) gene; or the Rosa26 locus/human orthologue of the Rosa26 locus.

32. The engineered T cell of any one of claims 1 to 29, wherein the chimeric antigen receptor is encoded by a nucleic acid introduced into an endogenous T cell receptor locus in the T cell's genome.

33. The engineered T cell of any one of claims 1 to 32, wherein the antigen-binding domain comprises an antibody, an antibody fragment, an scFv, a Fv, a Fab, a (Fab′)2, a single domain antibody (SDAB), a VH or VL domain, or a camelid VHH domain.

34. The engineered T cell of any one of claims 6 to 33, wherein the antigen expressed by the cancer cell is selected from the group consisting of B cell maturation antigen (BCMA), CD10, CD19, CD20, CD22, CD30, CD33, CD34, CD38, CD56, CD80/86, CD117, CD123, CD123, CD133, CD138, carcinoembryonic antigen (CEA), CS1/CD319/SLAMF7, disialoganglioside (GD2), EphA2, epidermal growth factor receptor (EGFR), epithelial cell adhesion molecule (EpCAM), glypican-3 (GPC3), HER2, integrin β7, Lewis-Y, mesothelin, MUC1, PD-L1, and Prostate Stem Cell Antigen (PSCA).

35. The engineered T cell of any one of claims 1 to 34, wherein the transmembrane domain comprises a transmembrane domain of a protein selected from the group consisting of the alpha chain of the T-cell receptor, the beta chain of the T-cell receptor, or the zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDI Ia, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDI Ib, ITGAX, CDIIc, ITGBI, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a combination thereof.

36. The engineered T cell of any one of claims 1 to 35, wherein the intracellular domain comprises a costimulatory domain comprising a portion of the intracellular domain of CD27, CD28, 4-IBB, OX40, CD30, CD40, PD-1, ICOS, lymphocyte function-associated antigen-1 (LFA-1), CD2, CD7, LIGHT, NKG2C, B7-H3, or a ligand that specifically binds CD83, or a combination thereof.

37. The engineered T cell of claim 36, wherein the portion of the intracellular domain is from CD28 and/or 4-1BB.

38. The engineered T cell of any one of claims 1 to 37, wherein the intracellular domain comprises a signaling domain from CD3-ζ.

39. The engineered T cell of any one of claims 1 to 38, wherein the engineered T cell is derived from an allogeneic T cell, an autologous T cell, or a tumor-infiltrating lymphocyte (TIL).

40. The engineered T cell of claim 39, wherein the engineered T cell is a CD4+ T cell.

41. The engineered T cell of claim 39, wherein the engineered T cell is a CD8+ T cell.

42. The engineered T cell of any one of claims 1 to 41, wherein the engineered T cell is an in vitro cell.

43. The engineered T cell of any one of claims 1 to 42, wherein the engineered T cell is a human cell.

44. The engineered T cell of any one of claims 1 to 43, for use as a medicament in the treatment of a cancer.

45. Use of the engineered T cell of any one of claims 1 to 44 in the manufacture of a medicament.

46. A composition comprising the engineered T cell of any one of claims 1 to 44.

47. The composition of claim 46, further comprising an engineered T cell that expresses a chimeric antigen receptor and does not express a heterologous chimeric protein.

48. The composition of claim 47, further comprising an engineered T cell that expresses a heterologous chimeric protein and does not expresses a chimeric antigen receptor.

49. The composition of claim 46, further comprising an engineered T cell that expresses a heterologous chimeric protein and does not expresses a chimeric antigen receptor.

50. The composition of any one of claims 46 to 49, further comprising a T cell that does not express a heterologous chimeric protein nor a chimeric antigen receptor.

51. A pharmaceutical composition comprising the composition of any one of claims 46 to 50 and a pharmaceutically acceptable excipient.

52. A method for manufacturing an engineered T cell, the method comprising steps of:
(i) obtaining a T-cell or T-cell progenitor cell from a subject;
(ii) transfecting the T-cell or T-cell progenitor cell with a first DNA molecule encoding a chimeric antigen receptor and designed to be integrated into the genome of the T cell or T-cell progenitor; and
(iii) transfecting the T-cell or T-cell progenitor cell with a second DNA molecule encoding a heterologous chimeric protein and designed to be integrated into the genome of the T cell or T-cell progenitor, wherein the heterologous chimeric protein comprises the general structure:
N terminus-(a)-(b)-(c)-C terminus,
wherein:
(a) is a first domain comprising an extracellular domain of a first transmembrane protein,
(b) is a linker domain adjoining the first and second domains, and
(c) is a second domain comprising an extracellular domain of a second transmembrane protein.

53. The method of claim 52, wherein step (ii) precedes step (iii).

54. The method of claim 52, wherein step (iii) precedes step (ii).

55. The method of claim 52, wherein step (ii) and step (iii) are contemporaneous.

56. A method for manufacturing an engineered T cell, the method comprising steps of:
(i) obtaining a T-cell or T-cell progenitor cell that has been transfected with a first DNA molecule encoding a chimeric antigen receptor and designed to be integrated into the genome of the T cell or T-cell progenitor; and
(ii) transfecting the T cell or T-cell progenitor cell of step (i) with a second DNA molecule encoding a heterologous chimeric protein and designed to be integrated into the genome of the T cell or T-cell progenitor, wherein the heterologous chimeric protein comprises the general structure:
N terminus-(a)-(b)-(c)-C terminus,
wherein:
(a) is a first domain comprising an extracellular domain of a first transmembrane protein,
(b) is a linker domain adjoining the first and second domains, and
(c) is a second domain comprising an extracellular domain of a second transmembrane protein.

57. A method for manufacturing an engineered T cell, the method comprising steps of:
(i) obtaining a T-cell or T-cell progenitor cell that has been transfected with a first DNA molecule encoding a heterologous chimeric protein and designed to be integrated into the genome of the T cell or T-cell progenitor, wherein the heterologous chimeric protein comprises the general structure:
N terminus-(a)-(b)-(c)-C terminus,
wherein:
(a) is a first domain comprising an extracellular domain of a first transmembrane protein,
(b) is a linker domain adjoining the first and second domains, and
(c) is a second domain comprising an extracellular domain of a second transmembrane protein; and
(ii) transfecting the T cell or T-cell progenitor cell of step (i) with a second DNA molecule encoding a chimeric antigen receptor and designed to be integrated into the genome of the T cell or T-cell progenitor.

58. The method of any one of claims 52 to 57, wherein the linker domain comprises at least one cysteine residue capable of forming a disulfide bond and/or comprises a hinge-CH2-CH3 Fc domain.

59. The method of any one of claims 52 to 58, wherein the first transmembrane protein is selected from the group consisting of PD-1, CSF1R, TIM-3, BTLA, CTLA-4, LAG-3, B7-H3, TMIGD2, TIGIT, CD172a/SIRPα, BTNL3, BTNL8, BTNL3A1, BTNL3A2, and VSIG8.

60. The method of any one of claims 52 to 59, wherein the second transmembrane protein is selected from the group consisting of OX40 ligand (OX40L), CD40 ligand (CD40L), 4-1BB ligand (4-1BBL), GITR ligand (GITRL), LIGHT (CD258), CD70, CD30 ligand, TRAIL, and T1A.

61. The method of any one of claims 52 to 60, wherein the DNA molecule encoding the chimeric antigen receptor is designed to be integrated into the genome of the T cell or T-cell progenitor at a genomic safe harbor (GSR).

62. The method of claim 61, wherein the GSR is the adeno-associated virus site 1 (AAVS1); the chemokine (C-C motif) receptor 5 (CCR5) gene; or the Rosa26 locus/human orthologue of the Rosa26 locus.

63. The method of any one of claims 52 to 60, wherein the DNA molecule encoding the chimeric antigen receptor is designed to be integrated into the genome of the T cell or T-cell progenitor at an endogenous T cell receptor locus.

64. The method of any one of claims 52 to 63, wherein the transfecting comprises addition of a zinc-finger nuclease, a meganuclease, a transcription activator-like effector (TALE) nuclease, or components of a CRISPR-Cas9 system to integrate the first DNA molecule and/or the second DNA molecule into the genome of the T cell or T-cell progenitor.

65. The method of any one of claims 52 to 64 further comprising a step of culturing the transfected T cell or T-cell progenitor cell in a medium that selectively enhances proliferation of chimeric antigen receptor-expressing cells and/or heterologous chimeric protein-expressing cells.

66. The method claim 65, further comprising a step of isolating an engineered T cell that expresses the chimeric antigen receptor and/or the heterologous chimeric protein.

67. An engineered T cell isolated by the method claim 66.

68. A method for obtaining a population of engineered T cells, the method comprising:
obtaining the engineered T cell of claim 67; and
culturing the engineered T cell in a medium that enhances proliferation of the engineered T cell, thereby obtaining a population of engineered T cells.

69. A population of engineered T cells obtained by the method of claim 68.

70. A pharmaceutical composition comprising the population of engineered T cells of claim 69 and a pharmaceutically acceptable excipient.

71. A method for treating a cancer in a subject in need thereof, the method comprising administering to the subject a therapeutically-effective amount of the pharmaceutical composition of claim 70.

72. The method of claim 71, wherein the T cell or T-cell progenitor was obtained from the subject.

73. A method for treating a cancer in a subject in need thereof, the method comprising administering to the subject a therapeutically effective amount of the pharmaceutical composition of claim 51.

74. The method of claim 73, wherein at least one engineered T cell in the pharmaceutical composition is allogenic.

75. The method of claim 74, wherein at least one engineered T cell in the pharmaceutical composition is autologous.

76. The method of claim 73, wherein at least one engineered T cell in the pharmaceutical composition is autologous.

77. The method of any one of claims 71 to 76, wherein the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers, or a combination thereof.

78. The method of any one of claims 71 to 76, wherein the cancer a hematologic cancer selected from the group consisting of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia, or a combination thereof.

79. The method of any one of claims 71 to 78, wherein the first transmembrane protein of the heterologous chimeric protein comprises PD-1 and the second transmembrane protein of the heterologous chimeric protein comprises OX40L.

80. The method of any one of claims 71 to 78, wherein the first transmembrane protein of the heterologous chimeric protein comprises PD-1 and the second transmembrane protein of the heterologous chimeric protein comprises 4-1BBL.

81. The method of any one of claims 71 to 78, wherein the first transmembrane protein of the heterologous chimeric protein comprises CSF1R and the second transmembrane protein of the heterologous chimeric protein comprises CD40L.

82. The method of any one of claims 71 to 81 further comprising administering to the subject in need thereof a pharmaceutical composition comprising the heterologous chimeric protein.

83. The method of claim 82, wherein the subject is administered the pharmaceutical composition comprising the heterologous chimeric protein before the subject is administered the pharmaceutical composition comprising the population of engineered T cells.

84. The method of claim 82, wherein the subject is administered the pharmaceutical composition comprising the heterologous chimeric protein after the subject is administered the pharmaceutical composition comprising the population of engineered T cells.

85. The method of claim 82, wherein the subject is administered the pharmaceutical composition comprising the heterologous chimeric protein contemporaneous with administering the pharmaceutical composition comprising the population of engineered T cells.

86. The method of any one of claims 82 to 85, wherein the dosage of the pharmaceutical composition comprising the heterologous chimeric protein is less than the dosage administered to a subject who has not or will not be administered the pharmaceutical composition comprising the population of engineered T cells.

87. The method of any one of claims 71 to 86 further comprising administering to the subject a therapeutically-effective amount an immunosuppressive drug.

88. The method of claim 87, wherein the immunosuppressive drug reduces or prevents Cytokine release syndrome.

89. The method of claim 88, wherein the immunosuppressive drug is an antibody directed against the interleukin-6 receptor (IL-6R).

90. The method of claim 89, wherein the antibody is Tocilizumab.

91. The method of any one of claims 87 to 90, wherein the immunosuppressive drug is administered before the pharmaceutical composition comprising the population of engineered T cells.

92. The method of any one of claims 87 to 90, wherein the immunosuppressive drug is administered after the pharmaceutical composition comprising the population of engineered T cells.

93. The method of any one of claims 87 to 90, wherein the immunosuppressive drug is administered contemporaneous with the pharmaceutical composition comprising the population of engineered T cells.
